SEQUENCE LISTING (29 sequences)

SEQ ID NO: 1 - DNA, 1911 bp, Bacillus thuringiensis
  CDS (1)...(1911)
atgttattatataacccaatatttcaaggaggattttatatgaataatgtattgaatagcgaaagaactaataagtgtgatgcgtataacgtagtggcccatgatccatttagttttgagcataaatcattagataccatacagcaagaatggatggagtggaaaagaaccgatcatagtttatatgtatctcctattgtgggaactatagctagttttctgctaaagaaaatagcggggcttataggaaaaagaatattaagtgagttaaagaatttaatttttcctagtggtagtatagaatcaatgcaagatattttaagaggggcagaacaattcctaaatcaaagacttgatgcagacacctttgctcgggtagaggcagaattgatagggcttcaagcaaatgtagaggaatttaatcaacaagtggacaattttttaaacccaaatcaaaaccctgttcctttagcaataattgattcggttaatacaatgcaacaattattcctaagtagattaccccagttccagatacaacgctatcagctattattattacctttatttgcacaagcagccaatttacaccttacctttattagagatgttattcttaatgcagatgaatggggaataccagcagcaacagtgcgcacatatagagagcacctaaaaagatatacacgcgattattccaattattgtataaacacgtaccaaactgctttccgaggtttaaacactcgtttacatgatatgttagagtttagaacatttatgtttttaaatgtattagactatgtatctatctggtcgttgtttaaatatcaaagtctgatggttacttcaagtgctaatttatatgcttcgggaagtggtagtaatcaaccttttactgcacaagactggccatttttatattctcttttccaagtgaattcaaattatataatgtctaattttggtggtaaccgagagactgctagttttggtgttcctattctggggggattcataataaattttttacttagttttagggttaattatactggaggagtttcatctggtctcctaggtgttgaaggaatttcaaacaactttaattgcaactcctctttatcaacaccagttgtaagaagttggctagattcaggtgtatatcgaggtgacctgcaacacaattggcgaacagacatctttatgaggactaatattgtaccttgtggtgcttttctattatctcttgctatgtttccagatgttaaaagtaattattttcctgattatttcattcgtaacatttccggaattattcgaaatattgataacatgaatttgagtagaccattacactttaatgaagtaagagatttaagagacactgaagttgctactttagtatctgtgcataatagaaaaaataatatctatgctgctcatgaaaatggtactatgattcattttgcgccggaaggttatacaggtttcacaatatcaccaatatatgcaactcaagtaaataatcaaacacgaacgtttatttctgaaaaattcggaaatcaaggtgattccttgagatttgaacaaactaacacaacggctcgttatacgtttagagggaatggtaataattataatctttatttaagagtatcctcacaaggaaattctacttttcgagttactataaacggtagggtttatactgtttcaaatgttaataccactacaaataatgatggggttattgataatggggctcgtttttcagatattcacatcgggaatatagtggcaagtaacaatactaatgtaccattagatataaatgtgatacttaactccggtactcaatttgagcttatgaatattatttttgttccaactaacattccaccactttat

SEQ ID NO: 2 - protein, 637 aa, Bacillus thuringiensis
MLLYNPIFQGGFYMNNVLNSERTNKCDAYNVVAHDPFSFEHKSLDTIQQEWMEWKRTDHSLYVSPIVGTIASFLLKKIAGLIGKRILSELKNLIFPSGSIESMQDILRGAEQFLNQRLDADTFARVEAELIGLQANVEEFNQQVDNFLNPNQNPVPLAIIDSVNTMQQLFLSRLPQFQIQRYQLLLLPLFAQAANLHLTFIRDVILNADEWGIPAATVRTYREHLKRYTRDYSNYCINTYQTAFRGLNTRLHDMLEFRTFMFLNVLDYVSIWSLFKYQSLMVTSSANLYASGSGSNQPFTAQDWPFLYSLFQVNSNYIMSNFGGNRETASFGVPILGGFIINFLLSFRVNYTGGVSSGLLGVEGISNNFNCNSSLSTPVVRSWLDSGVYRGDLQHNWRTDIFMRTNIVPCGAFLLSLAMFPDVKSNYFPDYFIRNISGIIRNIDNMNLSRPLHFNEVRDLRDTEVATLVSVHNRKNNIYAAHENGTMIHFAPEGYTGFTISPIYATQVNNQTRTFISEKFGNQGDSLRFEQTNTTARYTFRGNGNNYNLYLRVSSQGNSTFRVTINGRVYTVSNVNTTTNNDGVIDNGARFSDIHIGNIVASNNTNVPLDINVILNSGTQFELMNIIFVPTNIPPLY

SEQ ID NO: 3 - DNA, 1875 bp, Artificial Sequence
  Synthetic sequence encoding AXMI-066 (optaxmi-066)
atgaacaatgtgctcaacagcgagaggaccaacaaatgtgatgcctacaatgtggtggctcatgatcccttcagcttcgagcacaagagcttggacaccatccagcaagaatggatggagtggaagaggacagatcacagcctctatgtttcaccaattgttggcaccatcgccagcttcctgctgaagaagatcgccggcctcatcggcaagaggattctttctgagctgaagaacctcatcttcccatcaggaagcatcgagagcatgcaagacatcctccgcggcgccgagcagttcctcaaccagaggctggatgctgacaccttcgccagggtggaagctgagctcatcggccttcaagcaaatgtggaggagttcaaccagcaggtggacaacttcctcaaccccaaccagaacccggtgccgctggccatcattgattctgtgaacaccatgcagcagctcttcctctcaaggctgccgcagttccagatccaaagataccagctgctgctgctgccgctctttgctcaagctgccaacctccacctcaccttcatcagagatgtcatcctcaatgctgatgaatggggcatcccggcggcgacggtgaggacctacagggagcacctcaagagatacacaagggactactcaaactactgcatcaacacctaccagacggccttccgcggcctcaacacaaggctacatgacatgctggagttcagaaccttcatgttcctcaatgtgctggactatgtgagcatctggagcctcttcaagtaccagagcttgatggtgacaagctccgccaacctctatgcttctggaagcggcagcaaccagcccttcaccgctcaagattggcccttcctctacagcctcttccaagtgaactcaaactacatcatgagcaacttcggcggcaaccgtgaaactgcttccttcggcgtgcccatcctcggcggcttcatcatcaacttcctgctgagcttccgcgtcaactacactggaggagtttcttctgggctgctgggagttgaaggcatcagcaacaacttcaactgcaactcaagcctcagcacgccggtggtgaggagctggctggacagcggcgtctacagaggagatcttcagcacaactggaggacggacatcttcatgaggaccaacatcgtgccatgcggcgccttcctcctctcattggccatgttccctgatgtgaagagcaactacttcccggactacttcatcaggaacatcagcggcatcatcaggaacatcgacaacatgaacctctcaaggccgctgcacttcaacgaggtgagagatcttcgagacaccgaggtggccaccttggtgagcgtccacaacaggaagaacaacatctatgctgctcatgaaaatggcaccatgatccacttcgcgccagaaggctacaccggcttcaccatctcaccaatttatgcaactcaagtcaacaaccaaacaaggaccttcatctccgagaagtttggaaatcaaggagattctttgagatttgagcagaccaacaccacggcgcgctacaccttcagaggaaatggcaacaactacaacctctacctccgcgtcagcagccaaggcaacagcaccttccgcgtgaccatcaatggccgcgtctacaccgtctcaaatgtcaacaccaccaccaacaatgatggcgtcatcgacaatggagcaagattctccgacatccacatcggcaacatcgtcgccagcaacaacaccaacgtgccgctggacatcaatgtcatcctcaacagcggcacccaatttgagctgatgaacatcatcttcgtgccaaccaacatcccgccgctctactag

SEQ ID NO: 4 - DNA, 1902 bp, Bacillus thuringiensis
  CDS (1)...(1902)
atgaataatgtattaaataacggaagaactactaattgtgatgcgtataatgtagtggcccatgatccatttagttttgagcataaatcattagataccatccgaaaagaatggatggagtggaaaagaacagatcatagtttatatgtagctcctgtagtcggaactgtttctagctttctgctaaagaaggtggggagtcttattggaaaaaggatattgagtgaattatgggggttaatatttcctagtggtagcacaaatctaatgcaagatattttaagggagacagaacaattcctaaatcaaagacttaatacagacactcttgctcgtgtaaatgcggaattgacagggctgcaagcgaatataagggagtttaatcaacaagtagataattttttaaatcctactcaaaaccctgttcctttatcaataacttcttcagttaatacaatgcagcaattatttctaaatagattaccccagttccagatacaaggataccaactgttattattacctttatttgcacaggcagccaatatgcatctttcttttattagagatgttattcttaatgcagatgaatggggaatttcagcagcaacactacgtacgtatcgagactacctgagaaattatacaagagattattctaattattgtataaatacgtatcaaactgcgtttagagggttaaacacccgtttacacgatatgttagaatttagaacatatatgtttttaaatgtatttgaatatgtatccatttggtcattgtttaaatatcagagtcttatggtatcttctggcgctaatttatatgctagtggtagtggaccacagcagacccaatcatttatttcacaagactggccatttttatattctcttttccaagttaattcaaattatgtgttaaatggctttagtggcgctaggaatactattagattcccagctggtggtggtttattaccacctggtgttactacaactcacgcattgcttgctgcaagggtcaattacagtggaggagtttcgtctggttatataggcgctgtgtttaatcaaaattttaattgtagcacacttctcccacctttgttaacaccatttgttaggagttggctagattcaggtacagatcgggagggcgttaccaccgttacaaattggcaaacagaatcctttaagacaactttaggtttaaggtgtggtgcttttacaccccgtggtaattcaaactatttcccagattattttatccgtaatatttctggcgttcctttagttgttagaaacgaagatttaagaagaccgttacactataatgaaataagaaatatagaaagtccctcaggaacacctggtggattacgagcttatatggtatctgtgcataacagaaaaaataatatctatgccgctcatgaaaatggtactatgattcatttggcaccggaagattatacaggatttactatgttgccgatacatgcaactcaagtgaataatcaaacgcgaacatttatttctgaaaaatttggaaatcaaggtgattccttaagatttgaacaaagcgacacgacagctcgttatacacttagagggaatggaaatagttacaatctttatttaagagtatcttctctaggaaattccactattcgagttactataaacggaagagtttatactgttccaaatgttaatacaaatataaataacgatggagtcattgataatggagctcgtttttcagatattaatatcggtaatgtagtagcaagtgataatactaatgtaccgttagatataaacgtgacattaagttctggaactcaatttgagcttatgaatattatgtttgttccaactaatcttccaccaatatat

SEQ ID NO: 5 - protein, 634 aa, Bacillus thuringiensis
MNNVLNNGRTTNCDAYNVVAHDPFSFEHKSLDTIRKEWMEWKRTDHSLYVAPVVGTVSSFLLKKVGSLIGKRILSELWGLIFPSGSTNLMQDILRETEQFLNQRLNTDTLARVNAELTGLQANIREFNQQVDNFLNPTQNPVPLSITSSVNTMQQLFLNRLPQFQIQGYQLLLLPLFAQAANMHLSFIRDVILNADEWGISAATLRTYRDYLRNYTRDYSNYCINTYQTAFRGLNTRLHDMLEFRTYMFLNVFEYVSIWSLFKYQSLMVSSGANLYASGSGPQQTQSFISQDWPFLYSLFQVNSNYVLNGFSGARNTIRFPAGGGLLPPGVTTTHALLAARVNYSGGVSSGYIGAVFNQNFNCSTLLPPLLTPFVRSWLDSGTDREGVTTVTNWQTESFKTTLGLRCGAFTPRGNSNYFPDYFIRNISGVPLVVRNEDLRRPLHYNEIRNIESPSGTPGGLRAYMVSVHNRKNNIYAAHENGTMIHLAPEDYTGFTMLPIHATQVNNQTRTFISEKFGNQGDSLRFEQSDTTARYTLRGNGNSYNLYLRVSSLGNSTIRVTINGRVYTVPNVNTNINNDGVIDNGARFSDINIGNVVASDNTNVPLDINVTLSSGTQFELMNIMFVPTNLPPIY

SEQ ID NO: 6 - DNA, 1902 bp, Artificial Sequence
  Synthetic sequence encoding AXMI-076 (optaxmi-076)
atgaacaatgtgctcaacaatggaagaacaacaaattgtgatgcctacaatgtggtggctcatgatcccttcagcttcgagcacaagagcttggacaccatcaggaaggagtggatggaatggaagaggacagatcattctctttatgtggcgccggtggtgggcaccgtctcatccttcctgctgaagaaggtgggcagcctcatcggcaagaggattctttctgagctctggggcctcatcttcccaagtggatcaacaaatttgatgcaagacatcctccgagaaacagagcagttcctcaatcaaaggctcaacaccgacaccttggcaagggtgaatgctgagctgacagggctacaagccaacatcagggagttcaaccagcaggtggacaacttcctcaacccaactcaaaatccagtgccgctgagcatcacctcctctgttaacaccatgcagcagctcttcctcaacaggctgccgcagttccagatccaaggctaccagctgctgctgctgccgctatttgctcaagctgccaacatgcacctcagcttcatcagagatgtcatcctcaatgctgatgaatggggcatctcggcggcgacgctgaggacctacagagattatttgaggaactacacaagggactactcaaactactgcatcaacacctaccagacggccttccgcggcctcaacacaaggctgcatgacatgctggagttcagaacctacatgttcctcaatgtttttgaatatgtttcaatttggagcctcttcaagtaccagagcttgatggtgagcagcggcgccaacctctatgcttctggaagtgggccgcagcaaactcagagcttcatcagccaagattggcccttcctctacagcctcttccaagttaacagcaactacgtgctgaatggcttctcaggagcaaggaacaccatcagatttcctgctggaggagggctgctgccgccgggcgtgacaacaactcatgctctgctggcggcaagagttaactactctggaggagtttcaagcggctacatcggcgccgtcttcaaccagaacttcaactgctcgacgctgctgccgccgctgctgacgccatttgtgaggagctggctggacagcggcaccgacagagaaggagtcaccaccgtcaccaactggcaaactgagagcttcaagacgacgctgggcctccgctgcggcgccttcacgccgcgcggcaactcaaactacttcccggactacttcatcaggaacatcagcggcgtgccgctggtggtgaggaatgaagatttgaggaggccgctgcactacaatgagatcaggaacatcgagagcccatcaggaactcctggaggcctccgcgcctacatggtttctgttcacaacaggaagaacaacatctatgctgctcatgaaaatggcaccatgatccacctggcgccggaggactacaccggcttcaccatgctacccatccatgcaactcaagtcaacaaccaaacaaggaccttcatctccgagaagtttggaaatcaaggagattctttgagatttgagcagagcgacaccacggcgcgctacaccttgagaggaaatggcaacagctacaacctctacctccgcgtcagcagcctcggcaactcaaccatcagggtgaccatcaatggccgcgtctacaccgtgccaaatgtcaacaccaacatcaacaatgatggcgtcatcgacaatggagcaagattctccgacatcaacatcggcaatgtggtggcctccgacaacaccaacgtgccgctggacatcaatgtcaccttgagctcaggaactcaatttgagctgatgaacatcatgttcgtgccaacaaatcttcctcccatctac

SEQ ID NO: 7 - protein, 4 aa, Artificial Sequence
  Endoplasmic reticulum targeting sequence
KDEL

SEQ ID NO: 8 - DNA, 1990 bp, Bacillus thuringiensis
agacaggttcttaaacatacttgttattatcaagagagtattgtctctttaatgttattatataacccaatatttcaaggaggattttatatgaataatgtattgaatagcgaaagaactaataagtgtgatgcgtataacgtagtggcccatgatccatttagttttgagcataaatcattagataccatacagcaagaatggatggagtggaaaagaaccgatcatagtttatatgtatctcctattgtgggaactatagctagttttctgctaaagaaaatagcggggcttataggaaaaagaatattaagtgagttaaagaatttaatttttcctagtggtagtatagaatcaatgcaagatattttaagaggggcagaacaattcctaaatcaaagacttgatgcagacacctttgctcgggtagaggcagaattgatagggcttcaagcaaatgtagaggaatttaatcaacaagtggacaattttttaaacccaaatcaaaaccctgttcctttagcaataattgattcggttaatacaatgcaacaattattcctaagtagattaccccagttccagatacaacgctatcagctattattattacctttatttgcacaagcagccaatttacaccttacctttattagagatgttattcttaatgcagatgaatggggaataccagcagcaacagtgcgcacatatagagagcacctaaaaagatatacacgcgattattccaattattgtataaacacgtaccaaactgctttccgaggtttaaacactcgtttacatgatatgttagagtttagaacatttatgtttttaaatgtattagactatgtatctatctggtcgttgtttaaatatcaaagtctgatggttacttcaagtgctaatttatatgcttcgggaagtggtagtaatcaaccttttactgcacaagactggccatttttatattctcttttccaagtgaattcaaattatataatgtctaattttggtggtaaccgagagactgctagttttggtgttcctattctggggggattcataataaattttttacttagttttagggttaattatactggaggagtttcatctggtctcctaggtgttgaaggaatttcaaacaactttaattgcaactcctctttatcaacaccagttgtaagaagttggctagattcaggtgtatatcgaggtgacctgcaacacaattggcgaacagacatctttatgaggactaatattgtaccttgtggtgcttttctattatctcttgctatgtttccagatgttaaaagtaattattttcctgattatttcattcgtaacatttccggaattattcgaaatattgataacatgaatttgagtagaccattacactttaatgaagtaagagatttaagagacactgaagttgctactttagtatctgtgcataatagaaaaaataatatctatgctgctcatgaaaatggtactatgattcattttgcgccggaaggttatacaggtttcacaatatcaccaatatatgcaactcaagtaaataatcaaacacgaacgtttatttctgaaaaattcggaaatcaaggtgattccttgagatttgaacaaactaacacaacggctcgttatacgtttagagggaatggtaataattataatctttatttaagagtatcctcacaaggaaattctacttttcgagttactataaacggtagggtttatactgtttcaaatgttaataccactacaaataatgatggggttattgataatggggctcgtttttcagatattcacatcgggaatatagtggcaagtaacaatactaatgtaccattagatataaatgtgatacttaactccggtactcaatttgagcttatgaatattatttttgttccaactaacattccaccactttattaaggtttgagtttctgaggtaaatata

SEQ ID NO: 9 - DNA, 1872 bp, Bacillus thuringiensis
  CDS (1)...(1872)
atgaataatgtattgaatagcgaaagaactaataagtgtgatgcgtataacgtagtggcccatgatccatttagttttgagcataaatcattagataccatacagcaagaatggatggagtggaaaagaaccgatcatagtttatatgtatctcctattgtgggaactatagctagttttctgctaaagaaaatagcggggcttataggaaaaagaatattaagtgagttaaagaatttaatttttcctagtggtagtatagaatcaatgcaagatattttaagaggggcagaacaattcctaaatcaaagacttgatgcagacacctttgctcgggtagaggcagaattgatagggcttcaagcaaatgtagaggaatttaatcaacaagtggacaattttttaaacccaaatcaaaaccctgttcctttagcaataattgattcggttaatacaatgcaacaattattcctaagtagattaccccagttccagatacaacgctatcagctattattattacctttatttgcacaagcagccaatttacaccttacctttattagagatgttattcttaatgcagatgaatggggaataccagcagcaacagtgcgcacatatagagagcacctaaaaagatatacacgcgattattccaattattgtataaacacgtaccaaactgctttccgaggtttaaacactcgtttacatgatatgttagagtttagaacatttatgtttttaaatgtattagactatgtatctatctggtcgttgtttaaatatcaaagtctgatggttacttcaagtgctaatttatatgcttcgggaagtggtagtaatcaaccttttactgcacaagactggccatttttatattctcttttccaagtgaattcaaattatataatgtctaattttggtggtaaccgagagactgctagttttggtgttcctattctggggggattcataataaattttttacttagttttagggttaattatactggaggagtttcatctggtctcctaggtgttgaaggaatttcaaacaactttaattgcaactcctctttatcaacaccagttgtaagaagttggctagattcaggtgtatatcgaggtgacctgcaacacaattggcgaacagacatctttatgaggactaatattgtaccttgtggtgcttttctattatctcttgctatgtttccagatgttaaaagtaattattttcctgattatttcattcgtaacatttccggaattattcgaaatattgataacatgaatttgagtagaccattacactttaatgaagtaagagatttaagagacactgaagttgctactttagtatctgtgcataatagaaaaaataatatctatgctgctcatgaaaatggtactatgattcattttgcgccggaaggttatacaggtttcacaatatcaccaatatatgcaactcaagtaaataatcaaacacgaacgtttatttctgaaaaattcggaaatcaaggtgattccttgagatttgaacaaactaacacaacggctcgttatacgtttagagggaatggtaataattataatctttatttaagagtatcctcacaaggaaattctacttttcgagttactataaacggtagggtttatactgtttcaaatgttaataccactacaaataatgatggggttattgataatggggctcgtttttcagatattcacatcgggaatatagtggcaagtaacaatactaatgtaccattagatataaatgtgatacttaactccggtactcaatttgagcttatgaatattatttttgttccaactaacattccaccactttat

SEQ ID NO: 10 - protein, 624 aa, Bacillus thuringiensis
MNNVLNSERTNKCDAYNVVAHDPFSFEHKSLDTIQQEWMEWKRTDHSLYVSPIVGTIASFLLKKIAGLIGKRILSELKNLIFPSGSIESMQDILRGAEQFLNQRLDADTFARVEAELIGLQANVEEFNQQVDNFLNPNQNPVPLAIIDSVNTMQQLFLSRLPQFQIQRYQLLLLPLFAQAANLHLTFIRDVILNADEWGIPAATVRTYREHLKRYTRDYSNYCINTYQTAFRGLNTRLHDMLEFRTFMFLNVLDYVSIWSLFKYQSLMVTSSANLYASGSGSNQPFTAQDWPFLYSLFQVNSNYIMSNFGGNRETASFGVPILGGFIINFLLSFRVNYTGGVSSGLLGVEGISNNFNCNSSLSTPVVRSWLDSGVYRGDLQHNWRTDIFMRTNIVPCGAFLLSLAMFPDVKSNYFPDYFIRNISGIIRNIDNMNLSRPLHFNEVRDLRDTEVATLVSVHNRKNNIYAAHENGTMIHFAPEGYTGFTISPIYATQVNNQTRTFISEKFGNQGDSLRFEQTNTTARYTFRGNGNNYNLYLRVSSQGNSTFRVTINGRVYTVSNVNTTTNNDGVIDNGARFSDIHIGNIVASNNTNVPLDINVILNSGTQFELMNIIFVPTNIPPLY

SEQ ID NO: 11 - DNA, 1902 bp, Artificial Sequence
  Synthetic sequence encoding AXMI-076 (optaxmi-076v04)
atgaacaatgttctcaacaatggaagaacaacaaactgtgatgcctacaatgttgttgctcatgatcctttctcatttgaacacaagagcttggacaccatcagaaaagaatggatggaatggaaaagaacagatcattctctctatgttgctcctgttgttggaactgttagcagcttcttgctgaagaaggttggcagcttgattggaaaaaggattctttcagagctctggggcttgatctttccttctggaagcaccaacttgatgcaagacatcttgagagaaacagagcagttcttgaaccaaaggctcaacacagacaccttggcaagggtgaatgctgagctcactggccttcaagcaaacatcagagagttcaaccaacaagttgacaacttcttgaatccaacacaaaatcctgttcctctctccatcacttcttcagtgaacaccatgcagcagctcttcttgaacaggcttcctcagttccagattcaaggatatcagctgctgctgcttcctctctttgctcaagctgcaaacatgcatctctccttcatcagagatgtcatcttgaatgctgatgaatggggcatctctgctgccaccttgagaacatacagagattatttgagaaactacacaagagattattcaaactactgcatcaacacatatcaaacagctttcagaggcctcaacacaaggcttcatgacatgctggagttcagaacatacatgttcttgaatgtttttgaatatgtttccatctggagcttgttcaagtaccagagcttgatggtttcttctggagcaaatttatatgcatctggatctggacctcaacaaacacagagcttcatctctcaagattggccatttctctacagcttgttccaagtgaacagcaactatgttcttaatggcttctctggagcaagaaacaccatcagatttcctgctggaggaggcttgctgcctcctggtgtcaccaccacccatgctcttctggcggcgcgcgtcaactactcaggaggagtttcttctggatacattggagctgtgttcaaccaaaacttcaactgctccaccttgctgccgccgctgctgacaccatttgtcagaagctggctggactcaggaactgacagagaaggagtcaccaccgtcaccaactggcaaacagagagcttcaaaacaaccttgggcctccgctgcggcgccttcacgccgcgcggcaacagcaactactttccagattacttcatcagaaacatttctggagttcctttggtggtgagaaatgaagatctgaggaggcctcttcactacaatgaaatcagaaacattgaatctccatcaggaactcctggaggcctccgcgcctacatggtttctgttcacaacaggaagaacaacatctatgctgctcatgaaaatggaacaatgattcatcttgctccagaagattacactggcttcaccatgctgcccatccatgcaacacaagtcaacaaccaaacaaggaccttcatttcagaaaaatttggaaatcaaggagattctttgagatttgaacaaagcgacaccacagcaagatacaccttgagaggaaatggaaacagctacaacttgtacctccgcgtcagcagcttgggaaacagcaccatcagggtgaccatcaatggaagagtctacactgttccaaatgtcaacaccaacatcaacaatgatggtgtcattgacaatggagcaagattttcagacatcaacattggaaatgtggtggcttctgacaacacaaatgttcctctggacatcaatgtcaccctttcttctggaacacaatttgagctgatgaacatcatgtttgttccaacaaatcttcctccaatctac

SEQ ID NO: 12 - DNA, 2130 bp, Artificial Sequence
  Synthetic sequence encoding AXMI-076 with a chloroplast targeting peptide (optaxmi-076v04)
atgcagctgctgaaccaaagacaagctcttcgccttggaagaagctcagcaagcaaaaatcaacaagtggcgccgctggcatcaaggccagcttcttctctttctgtttctgcttcttctgtggcgccggcgccggcatgctctgctcctgctggagctggtagaagagctgtggtggtgagagcttctgcaacaaaggagaaggtggaggagctcaccatccagatgaacaatgttctcaacaatggaagaacaacaaactgtgatgcctacaatgttgttgctcatgatcctttctcatttgaacacaagagcttggacaccatcagaaaagaatggatggaatggaaaagaacagatcattctctctatgttgctcctgttgttggaactgttagcagcttcttgctgaagaaggttggcagcttgattggaaaaaggattctttcagagctctggggcttgatctttccttctggaagcaccaacttgatgcaagacatcttgagagaaacagagcagttcttgaaccaaaggctcaacacagacaccttggcaagggtgaatgctgagctcactggccttcaagcaaacatcagagagttcaaccaacaagttgacaacttcttgaatccaacacaaaatcctgttcctctctccatcacttcttcagtgaacaccatgcagcagctcttcttgaacaggcttcctcagttccagattcaaggatatcagctgctgctgcttcctctctttgctcaagctgcaaacatgcatctctccttcatcagagatgtcatcttgaatgctgatgaatggggcatctctgctgccaccttgagaacatacagagattatttgagaaactacacaagagattattcaaactactgcatcaacacatatcaaacagctttcagaggcctcaacacaaggcttcatgacatgctggagttcagaacatacatgttcttgaatgtttttgaatatgtttccatctggagcttgttcaagtaccagagcttgatggtttcttctggagcaaatttatatgcatctggatctggacctcaacaaacacagagcttcatctctcaagattggccatttctctacagcttgttccaagtgaacagcaactatgttcttaatggcttctctggagcaagaaacaccatcagatttcctgctggaggaggcttgctgcctcctggtgtcaccaccacccatgctcttctggcggcgcgcgtcaactactcaggaggagtttcttctggatacattggagctgtgttcaaccaaaacttcaactgctccaccttgctgccgccgctgctgacaccatttgtcagaagctggctggactcaggaactgacagagaaggagtcaccaccgtcaccaactggcaaacagagagcttcaaaacaaccttgggcctccgctgcggcgccttcacgccgcgcggcaacagcaactactttccagattacttcatcagaaacatttctggagttcctttggtggtgagaaatgaagatctgaggaggcctcttcactacaatgaaatcagaaacattgaatctccatcaggaactcctggaggcctccgcgcctacatggtttctgttcacaacaggaagaacaacatctatgctgctcatgaaaatggaacaatgattcatcttgctccagaagattacactggcttcaccatgctgcccatccatgcaacacaagtcaacaaccaaacaaggaccttcatttcagaaaaatttggaaatcaaggagattctttgagatttgaacaaagcgacaccacagcaagatacaccttgagaggaaatggaaacagctacaacttgtacctccgcgtcagcagcttgggaaacagcaccatcagggtgaccatcaatggaagagtctacactgttccaaatgtcaacaccaacatcaacaatgatggtgtcattgacaatggagcaagattttcagacatcaacattggaaatgtggtggcttctgacaacacaaatgttcctctggacatcaatgtcaccctttcttctggaacacaatttgagctgatgaacatcatgtttgttccaacaaatcttcctccaatctactaa

SEQ ID NO: 13 - protein, 709 aa, Artificial Sequence
  AXMI-076 with a chlorooplast targeting peptide
MQLLNQRQALRLGRSSASKNQQVAPLASRPASSLSVSASSVAPAPACSAPAGAGRRAVVVRASATKEKVEELTIQMNNVLNNGRTTNCDAYNVVAHDPFSFEHKSLDTIRKEWMEWKRTDHSLYVAPVVGTVSSFLLKKVGSLIGKRILSELWGLIFPSGSTNLMQDILRETEQFLNQRLNTDTLARVNAELTGLQANIREFNQQVDNFLNPTQNPVPLSITSSVNTMQQLFLNRLPQFQIQGYQLLLLPLFAQAANMHLSFIRDVILNADEWGISAATLRTYRDYLRNYTRDYSNYCINTYQTAFRGLNTRLHDMLEFRTYMFLNVFEYVSIWSLFKYQSLMVSSGANLYASGSGPQQTQSFISQDWPFLYSLFQVNSNYVLNGFSGARNTIRFPAGGGLLPPGVTTTHALLAARVNYSGGVSSGYIGAVFNQNFNCSTLLPPLLTPFVRSWLDSGTDREGVTTVTNWQTESFKTTLGLRCGAFTPRGNSNYFPDYFIRNISGVPLVVRNEDLRRPLHYNEIRNIESPSGTPGGLRAYMVSVHNRKNNIYAAHENGTMIHLAPEDYTGFTMLPIHATQVNNQTRTFISEKFGNQGDSLRFEQSDTTARYTLRGNGNSYNLYLRVSSLGNSTIRVTINGRVYTVPNVNTNINNDGVIDNGARFSDINIGNVVASDNTNVPLDINVTLSSGTQFELMNIMFVPTNLPPIY

SEQ ID NO: 14 - protein, 633 aa, Bacillus thuringiensis
MNNVLNSGRTTICDAYNVVAHDPFSFEHKSLDTIQKEWMEWKRTDHSLYVAPVVGTVSSFLLKKVGSLIGKRILSELWGIIFPSGSTNLMQDILRETEQFLNQRLNTDTLARVNAELIGLQANIREFNQQVDNFLNPTQNPVPLSITSSVNTMQQLFLNRLPQFQIQGYQLLLLPLFAQAANMHLSFIRDVILNADEWGISAATLRTYRDYLRNYTRDYSNYCINTYQTAFRGLNTRLHDMLEFRTYMFLNVFEYVSIWSLFKYQSLMVSSGANLYASGSGPQQTQSFTAQNWPFLYSLFQVNSNYILSGISGTRLSITFPNIGGLPGSTTTHSLNSARVNYSGGVSSGLIGATNLNHNFNCSTVLPPLSTPFVRSWLDSGTDREGVATSTNWQTESFQTTLSLRCGAFSARGNSNYFPDYFIRNISGVPLVIRNEDLTRPLHYNQIRNIESPSGTPGGARAYLVSVHNRKNNIYAANENGTMIHLAPEDYTGFTISPIHATQVNNQTRTFISEKFGNQGDSLRFEQSNTTARYTLRGNGNSYNLYLRVSSIGNSTIRVTINGRVYTVSNVNTTTNNDGVNDNGARFSDINIGNIVASDNTNVTLDINVTLNSGTPFDLMNIMFVPTNLPPLY

SEQ ID NO: 15 - protein, 633 aa, Bacillus thuringiensis
MNSVLNSGRTTICDAYNVAAHDPFSFQHKSLDTVQKEWTEWKKNNHSLYLDPIVGTVASFLLKKVGSLVGKRILSELRNLIFPSGSTNLMQDILRETEKFLNQRLNTDTLARVNAELTGLQANVEEFNRQVDNFLNPNRNAVPLSITSSVNTMQQLFLNRLPQFQMQGYQLLLLPLFAQAANLHLSFIRDVILNADEWGISAATLRTYRDYLKNYTRDYSNYCINTYQSAFKGLNTRLHDMLEFRTYMFLNVFEYVSIWSLFKYQSLLVSSGANLYASGSGPQQTQSFTSQDWPFLYSLFQVNSNYVLNGFSGARLSNTFPNIVGLPGSTTTHALLAARVNYSGGISSGDIGASPFNQNFNCSTFLPPLLTPFVRSWLDSGSDREGVATVTNWQTESFETTLGLRSGAFTARGNSNYFPDYFIRNISGVPLVVRNEDLRRPLHYNEIRNIASPSGTPGGARAYMVSVHNRKNNIHAVHENGSMIHLAPNDYTGFTISPIHATQVNNQTRTFISEKFGNQGDSLRFEQNNTTARYTLRGNGNSYNLYLRVSSIGNSTIRVTINGRVYTATNVNTTTNNDGVNDNGARFSDINIGNVVASSNSDVPLDINVTLNSGTQFDLMNIMLVPTNISPLY

SEQ ID NO: 16 - protein, 622 aa, Bacillus thuringiensis
MNTVLNNGRNTTCHAHNVVAHDPFSFEHKSLNTIEKEWKEWKRTDHSLYVAPIVGTVGSFLLKKVGSLVGKRILSELQNLIFPSGSIDLMQEILRATEQFINQRLNADTLGRVNAELAGLQANVAEFNRQVDNFLNPNQNPVPLAIIDSVNTLQQLFLSRLPQFQIQGYQLLLLPLFAQAANFNLSFIRGVILNADEWGISAATVRTYRDHLRKFHRDYSNYCINPYQTAFRGLNHRLPDMLEFRTYMFLNVFEYVSIWSLFKYQSLLVSSGANLYASGSGPTQSFTAQNWPFLYSLFQVNSNYVLNGLSGARTTITFPNIGGLPVYHNSTLHFARINYRGGVSSSRIGQANLNQNFNISTLFNPLQTPFIRSWLDSGTDREGVATSTNWQSGAFETTLLRFSIFSARGNSNFFPDYFIRNISGVVGTISNADLARPLHFNEIRDIGTTAVASLVTVHNRKNNIYDTHENGTMIHLAPNDYTGFTVSPIHATQVNNQIRTFISEKYGNQGDSLRFELSNPTARYTLRGNGNSYNLYLRVSSIGSSTIRVTINGRVYTANVNTTTNNDGVLDNGARFSDINIGNVVASANTNVPLDIQVTFNGNPQFELMNIMFVPTNLPPLY

SEQ ID NO: 17 - protein, 633 aa, Bacillus thuringiensis
MNSVLNSGRNTICDAYNVVVHDPFSFQHKSLDTIQKEWMEWKKDNHSLYVDPIVGTVASFLLKKLGSLIGKRILSELRNLIFPSGSTNLMEDILRETEKFLNQKLNTDTLSRVNAELTGLQANVEEFNRQVDNFLNPNRNAVPLSITSSVNTMQQLFLNRLSQFQMQGYQLLLLPLFAQAANLHLSFIRDVILNAEEWGISAATLRTYQNHLRNYTRDYSNYCIDTYQTAFRGLNTRLHDMLEFRTYMFLNVFEYVSIWSLFKYQSLLVSSGANLYASGSGPQQTQLFTSQDWPFLYSLFQVNSNYVLSGFSGASLFTTFPNIGGLPGSTTTQALLAARVNYSGGITSGSIGGSNFNQNFNCNTISPPLSTSFVRSWLDSGSDRQGVNTVTNWQTESFETTSGLRCGAFTPRGNSNYYPGYFIRNISGVSLVLRNEDLKRPLYYNEKRNIESPSGTPGGARAYMVSVHNKKNNIYAVHENGTMIHLAPEDNTGFTISPIHATQVNNQTRTFISEKFGNQGDSLRFEQSNTTARYTLRGNGNSYNLYLRVSSIGNSTIRVTINGRVYTASNVNTTTNNDGVNDNGARFSDINIGNVVASSNSDVPLDINVTLNSGTQFDLMNIMLVPTNISPLY

SEQ ID NO: 18 - protein, 632 aa, Bacillus thuringiensis
MNNVLNNGRTTICDAYNVVAHDPFSFEHKSLDTIRKEWMEWKRTDHSLYVAPIVGTVSSFLLKKVGSLIGKRILSELWGLIFPSGSTNLMQDILRETEQFLNQRLNTDTLARVNAELEGLQANIREFNQQVDNFLNPTQNPVPLSITSSVNTMQQLFLNRLPQFRVQGYQLLLLPLFAQAANMHLSFIRDVVLNADEWGISAATLRTYQNYLKNYTTEYSNYCINTYQTAFRGLNTRLHDMLEFRTYMFLNVFEYVSIWSLFKYQSLLVSSGANLYASGSGPQQTQSFTSQDWPFLYSLFQVNSNYVLNGFSGARLTQTFPNIGGLPGTTTTHALLAARVNYSGGVSSGDIGAVFNQNFSCSTFLPPLLTPFVRSWLDSGSDRGGVNTVTNWQTESFESTLGLRCGAFTARGNSNYFPDYFIRNISGVPLVVRNEDLRRPLHYNEIRNIESPSGTPGGLRAYMVSVHNRKNNIYAVHENGTMIHLAPEDYTGFTISPIHATQVNNQTRTFISEKFGNQGDSLRFEQSNTTARYTLRGNGNSYNLYLRVSSLGNSTIRVTINGRVYTASNVNTTTNNDGVNDNGARFLDINMGNVVASDNTNVPLDINVTFNSGTQFELMNIMFVPTNLPPIY

SEQ ID NO: 19 - protein, 1178 aa, Bacillus thuringiensis
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDYAVRWYNTGLERVWGPDSRDWVRYNQFRRELTLTVLDIVALFPNYDSRRYPIRTVSQLTREIYTNPVLENFDGSFRGSAQGIERSIRSPHLMDILNSITIYTDAHRGYYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIASDSITQIPAVKGNFLFNGSVISGPGFTGGDLVRLNSSGNNIQNRGYIEVPIHFPSTSTRYRVRVRYASVTPIHLNVNWGNSSIFSNTVPATATSLDNLQSSDFGYFESANAFTSSLGNIVGVRNFSGTAGVIIDRFEFIPVTATLEAEYNLERAQKAVNALFTSTNQLGLKTNVTDYHIDQVSNLVTYLSDEFCLDEKRELSEKVKHAKRLSDERNLLQDSNFKDINRQPERGWGGSTGITIQGGDDVFKENYVTLSGTFDECYPTYLYQKIDESKLKAFTRYQLRGYIEDSQDLEIYLIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRENPCEFNRGYRDYTPLPVGYVTKELEYFPETDKVWIEIGETEGTFIVDSVELLLMEE

SEQ ID NO: 20 - protein, 652 aa, Bacillus thuringiensis
MIRKGGRKMNPNNRSEHDTIKTTENNEVPTNHVQYPLAETPNPTLEDLNYKEFLRMTADNNTEALDSSTTKDVIQKGISVVGDLLGVVGFPFGGALVSFYTNFLNTIWPSEDPWKAFMEQVEALMDQKIADYAKNKALAELQGLQNNVEDYVSALSSWQKNPVSSRNPHSQGRIRELFSQAESHFRNSMPSFAISGYEVLFLTTYAQAANTHLFLLKDAQIYGEEWGYEKEDIAEFYKRQLKLTQEYTDHCVKWYNVGLDKLRGSSYESWVNFNRYRREMTLTVLDLIALFPLYDVRLYPKEVKTELTRDVLTDPIVGVNNLRGYGTTFSNIENYIRKPHLFDYLHRIQFHTRFQPGYYGNDSFNYWSGNYVSTRPSIGSNDIITSPFYGNKSSEPVQNLEFNGEKVYRAVANTNLAVWPSAVYSGVTKVEFSQYNDQTDEASTQTYDSKRNVGAVSWDSIDQLPPETTDEPLEKGYSHQLNYVMCFLMQGSRGTIPVLTWTHKSVDFFNMIDSKKITQLPLVKAYKLQSGASVVAGPRFTGGDIIQCTENGSAATIYVTPDVSYSQKYRARIHYASTSQITFTLSLDGAPFNQYYFDKTINKGDTLTYNSFNLASFSTPFELSGNNLQIGVTGLSAGDKVYIDKIEFIPVN

SEQ ID NO: 21 - DNA, 35 bp, Artificial Sequence
  oligonucleotide primer
cttccttcggcgtgnwnnwncccatcctcggcggc

SEQ ID NO: 22 - DNA, 35 bp, Artificial Sequence
  oligonucleotide primer
cttccttcggcgtgnsnnsncccatcctcggcggc

SEQ ID NO: 23 - DNA, 35 bp, Artificial Sequence
  oligonucleotide primer
cttccttcggcgtgnwnnsncccatcctcggcggc

SEQ ID NO: 24 - DNA, 35 bp, Artificial Sequence
  oligonucleotide primer
cttccttcggcgtgnsnnwncccatcctcggcggc

SEQ ID NO: 25 - DNA, 40 bp, Artificial Sequence
  oligonucleotide primer
cggcgtctacagagganwnnwngatcttcagcacaactgg

SEQ ID NO: 26 - DNA, 40 bp, Artificial Sequence
  oligonucleotide primer
cggcgtctacagaggansnnsngatcttcagcacaactgg

SEQ ID NO: 27 - DNA, 40 bp, Artificial Sequence
  oligonucleotide primer
cggcgtctacagaggansnnwngatcttcagcacaactgg

SEQ ID NO: 28 - DNA, 40 bp, Artificial Sequence
  oligonucleotide primer
cggcgtctacagagganwnnsngatcttcagcacaactgg

SEQ ID NO: 29 - DNA, 35 bp, Artificial Sequence
  oligonucleotide primer
cgccttcctcctctcagtgaagagcaactacttcc

(19) United States
(12) Patent Application Publication
(10) Pub. No.: US 2009/0144852 A1
(43) Pub. Date: Jun. 4, 2009

(54) AXMI-066 AND AXMI-076: DELTA-ENDOTOXIN PROTEINS AND METHODS FOR THEIR USE

(75) Inventors: Daniel J. Tomso, Bahama, NC (US); Nalini Desai, Chapel Hill, NC (US); Shruti Agarwal, Durham, NC (US); Kimberly S. Sampson, Durham, NC (US); Sandra Volrath, Durham, NC (US); Volker Heinrichs, Raleigh, NC (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Athenix Corporation, Research Triangle Park, NC (US)

(21) Appl. No.: 12/252,453

(22) Filed: Oct. 16, 2008

Related U.S. Application Data
(60) Provisional application No. 60/980,439, filed on Oct. 16, 2007.

(51) Int. Cl. C12N 15/82; A01H 5/00; C12N 15/11; C12N 15/00; C12N 1/21; C12N 5/04; C07K 14/00; A61K 38/16; C12P 21/02

(52) U.S. Cl. 800/278; 800/302; 536/23.1; 435/320.1; 435/252.3; 435/419; 530/350; 514/12; 435/69.1

(57) ABSTRACT

Compositions and methods for conferring pesticidal activity to bacteria, plants, plant cells, tissues and seeds are provided. Compositions comprising a coding sequence for pesticidal polypeptides are provided. The coding sequences can be used in DNA constructs or expression cassettes for transformation and expression in plants and bacteria. Compositions also comprise transformed bacteria, plants, plant cells, tissues, and seeds. In particular, isolated pesticidal nucleic acid molecules are provided. Additionally, amino acid sequences corresponding to the polynucleotides are encompassed. In particular, the present invention provides for isolated nucleic acid molecules comprising nucleotide sequences encoding the amino acid sequence shown in SEQ ID NO:5, 2, or 10, the nucleotide sequence set forth in SEQ ID NO:4, 1, 3, 4, 6, 9, or 11, or the nucleotide sequence deposited in a bacterial host as Accession No. B-50045, as well as variants and fragments thereof.

### CROSS-REFERENCE TO RELATED APPLICATIONS

This application claims the benefit of U.S. Provisional Application No. 60/980,439, filed Oct. 16, 2007, which is hereby incorporated in its entirety by reference herein.

### REFERENCE TO SEQUENCE LISTING SUBMITTED ELECTRONICALLY

The official copy of the sequence listing is submitted electronically via EFS-Web as an ASCII formatted sequence listing with a file named “363856_SequenceListing.txt”, created on Oct. 13, 2008, and having a size of 120 kilobytes and is filed concurrently with the specification. The sequence listing contained in this ASCII formatted document is part of the specification and is herein incorporated by reference in its entirety.

### FIELD OF THE INVENTION

This invention relates to the field of molecular biology. Provided are novel genes that encode pesticidal proteins. These proteins and the nucleic acid sequences that encode them are useful in preparing pesticidal formulations and in the production of transgenic pest-resistant plants.

### BACKGROUND OF THE INVENTION

Bacillus thuringiensis is a Gram-positive spore forming soil bacterium characterized by its ability to produce crystalline inclusions that are specifically toxic to certain orders and species of insects, but are harmless to plants and other non-targeted organisms. For this reason, compositions including Bacillus thuringiensis strains or their insecticidal proteins can be used as environmentally-acceptable insecticides to control agricultural insect pests or insect vectors for a variety of human or animal diseases.

Crystal (Cry) proteins (delta-endotoxins) from Bacillus thuringiensis have potent insecticidal activity against predominantly Lepidopteran, Dipteran, and Coleopteran larvae. These proteins also have shown activity against Hymenoptera, Homoptera, Phthiraptera, Mallophaga, and Acari pest orders, as well as other invertebrate orders such as Nemathelminthes, Platyhelminthes, and Sarcomastigorphora (Feitelson (1993) The Bacillus Thuringiensis family tree. In Advanced Engineered Pesticides, Marcel Dekker, Inc., New York, N.Y.) These proteins were originally classified as CryI to CryV based primarily on their insecticidal activity. The major classes were Lepidoptera-specific (I), Lepidoptera- and Diptera-specific (II), Coleoptera-specific (III), Diptera-specific (IV), and nematode-specific (V) and (VI). The proteins were further classified into subfamilies; more highly related proteins within each family were assigned divisional letters such as Cry1A, Cry1B, Cry1C, etc. Even more closely related proteins within each division were given names such as Cry1C1, Cry1C2, etc.

A new nomenclature was recently described for the Cry genes based upon amino acid sequence homology rather than insect target specificity (Crickmore et al. (1998) Microbiol. Mol. Biol. Rev. 62:807-813). In the new classification, each toxin is assigned a unique name incorporating a primary rank (an Arabic number), a secondary rank (an uppercase letter), a tertiary rank (a lowercase letter), and a quaternary rank (another Arabic number). In the new classification, Roman numerals have been exchanged for Arabic numerals in the primary rank. Proteins with less than 45% sequence identity have different primary ranks, and the criteria for secondary and tertiary ranks are 78% and 95%, respectively.

The crystal protein does not exhibit insecticidal activity until it has been ingested and solubilized in the insect midgut. The ingested protoxin is hydrolyzed by proteases in the insect digestive tract to an active toxic molecule. (Höfte and Whiteley (1989) Microbiol. Rev. 53:242-255). This toxin binds to apical brush border receptors in the midgut of the target larvae and inserts into the apical membrane creating ion channels or pores, resulting in larval death.

Delta-endotoxins generally have five conserved sequence domains, and three conserved structural domains (see, for example, de Maagd et al. (2001) Trends Genetics 17:193-199). The first conserved structural domain consists of seven alpha helices and is involved in membrane insertion and pore formation. Domain II consists of three beta-sheets arranged in a Greek key configuration, and domain III consists of two antiparallel beta-sheets in “jelly-roll” formation (de Maagd et al., 2001, supra). Domains II and III are involved in receptor recognition and binding, and are therefore considered determinants of toxin specificity.

Because of the devastation that insects can confer, and the improvement in yield by controlling insect pests, there is a continual need to discover new forms of pesticidal toxins.

### SUMMARY OF INVENTION

Compositions and methods for conferring pesticidal activity to bacteria, plants, plant cells, tissues and seeds are provided. Compositions include nucleic acid molecules encoding sequences for pesticidal and insectidal polypeptides, vectors comprising those nucleic acid molecules, and host cells comprising the vectors. Compositions also include the pesticidal polypeptide sequences and antibodies to those polypeptides. The nucleotide sequences can be used in DNA constructs or expression cassettes for transformation and expression in organisms, including microorganisms and plants. The nucleotide or amino acid sequences may be synthetic sequences that have been designed for expression in an organism including, but not limited to, a microorganism or a plant. Compositions also comprise transformed bacteria, plants, plant cells, tissues, and seeds.

In particular, isolated nucleic acid molecules are provided that encode a pesticidal protein. Additionally, amino acid sequences corresponding to the pesticidal protein are encompassed. In particular, the present invention provides for an isolated nucleic acid molecule comprising a nucleotide sequence encoding the amino acid sequence shown in SEQ ID NO:2 or 5, a nucleotide sequence set forth in SEQ ID NO:1, 3, 4, 6, 9, or 11, or the delta-endotoxin nucleotide sequence of the DNA insert of the plasmid deposited in a bacterial host as Accession No. B-50045, as well as variants and fragments thereof. Nucleotide sequences that are complementary to a nucleotide sequence of the invention, or that hybridize to a sequence of the invention are also encompassed.

Methods are provided for producing the polypeptides of the invention, and for using those polypeptides for controlling or killing a lepidopteran, coleopteran, nematode, or dipteran pest. Methods and kits for detecting the nucleic acids and polypeptides of the invention in a sample are also included.

The compositions and methods of the invention are useful for the production of organisms with enhanced pest resistance or tolerance. These organisms and compositions comprising the organisms are desirable for agricultural purposes. The compositions of the invention are also useful for generating altered or improved proteins that have pesticidal activity, or for detecting the presence of pesticidal proteins or nucleic acids in products or organisms.

### DESCRIPTION OF FIGURES

FIG. 1 shows the DNA sequence of the axmi-066 gene and its surrounding DNA region (SEQ ID NO:8). The first ATG (corresponding to the start site of SEQ ID NO:1; translation of which encodes AXMI-066 (SEQ ID NO:2)) is at nucleotide position 52 of the sequence shown in this figure. The second internal methionine (whose translation encodes residues 14 through 637 of SEQ ID NO:2) is at position 91 of this figure. The TAA stop codon begins at position 1963 of the sequence in this figure. The ATG start codons and the TAA stop codon are shown in bold type. Two putative ribosome binding sites are shown in italics and underlined.

FIGS. 2A-2D show an alignment of AXMI-066_long (SEQ ID NO:2), AXMI-066 (SEQ ID NO:10), Cry2Aa1 (SEQ ID NO:14), Cry2Ab1 (SEQ ID NO:15), Cry2Ac1 (SEQ ID NO:16), Cry2Ad1 (SEQ ID NO:17), Cry2Ae1 (SEQ ID NO:18), Cry1Ac (SEQ ID NO:19), and Cry3Aa1 (SEQ ID NO:20). The alignment shows the most highly conserved amino acid residues highlighted in black, and highly conserved amino acid residues highlighted in gray.

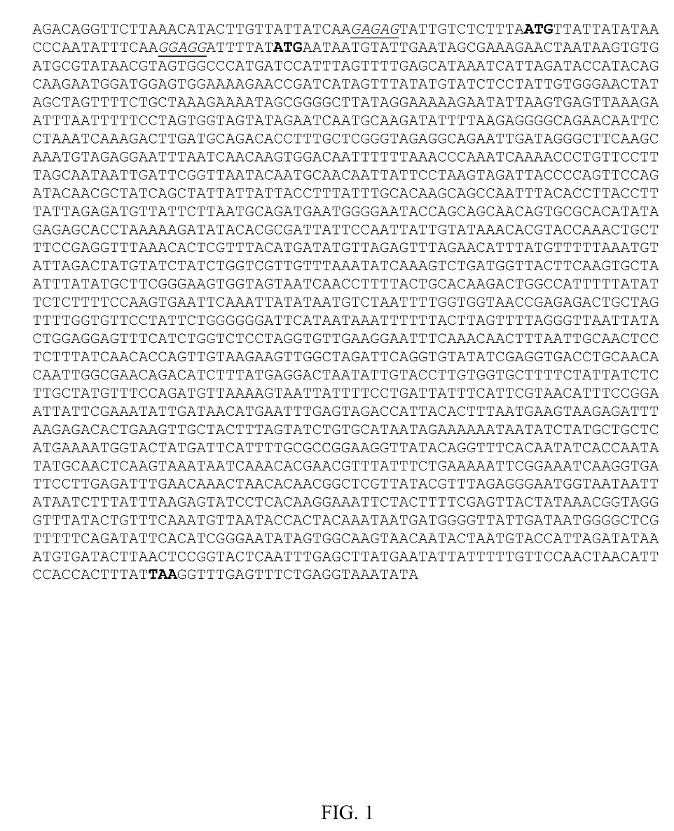

### DETAILED DESCRIPTION

The present invention is drawn to compositions and methods for regulating pest resistance or tolerance in organisms, particularly plants or plant cells. By “resistance” is intended that the pest (e.g., insect) is killed upon ingestion or other contact with the polypeptides of the invention. By “tolerance” is intended an impairment or reduction in the movement, feeding, reproduction, or other functions of the pest. The methods involve transforming organisms with a nucleotide sequence encoding a pesticidal protein of the invention. In particular, the nucleotide sequences of the invention are useful for preparing plants and microorganisms that possess pesticidal activity. Thus, transformed bacteria, plants, plant cells, plant tissues and seeds are provided. Compositions are pesticidal nucleic acids and proteins of Bacillus or other species. The sequences find use in the construction of expression vectors for subsequent transformation into organisms of interest, as probes for the isolation of other homologous (or partially homologous) genes, and for the generation of altered pesticidal proteins by methods known in the art, such as domain swapping or DNA shuffling. The proteins find use in controlling or killing lepidopteran, coleopteran, dipteran, and nematode pest populations and for producing compositions with pesticidal activity.

A plasmid containing the axmi-066 nucleotide sequence of the invention was deposited in the permanent collection of the Agricultural Research Service Culture Collection, Northern Regional Research Laboratory (NRRL), 1815 North University Street, Peoria, Ill. 61604, United States of America, on May 29, 2007. This deposit will be maintained under the terms of the Budapest Treaty on the International Recognition of the Deposit of Microorganisms for the Purposes of Patent Procedure. Access to these deposits will be available during the pendency of the application to the Commissioner of Patents and Trademarks and persons determined by the Commissioner to be entitled thereto upon request. Upon allowance of any claims in the application, the Applicants will make available to the public, pursuant to 37 C.F.R. § 1.808, sample(s) of the deposit with the NRRL. This deposit was made merely as a convenience for those of skill in the art and is not an admission that a deposit is required under 35 U.S.C. § 112.

By “pesticidal toxin” or “pesticidal protein” is intended a toxin that has toxic activity against one or more pests, including, but not limited to, members of the Lepidoptera, Diptera, and Coleoptera orders, or the Nematoda phylum, or a protein that has homology to such a protein. Pesticidal proteins have been isolated from organisms including, for example, Bacillus sp., Clostridium bifermentans and Paenibacillus popilliae. Pesticidal proteins include amino acid sequences deduced from the full-length nucleotide sequences disclosed herein, and amino acid sequences that are shorter than the full-length sequences, either due to the use of an alternate downstream start site, or due to processing that produces a shorter protein having pesticidal activity. Processing may occur in the organism the protein is expressed in, or in the pest after ingestion of the protein.

Pesticidal proteins encompass delta-endotoxins. Delta-endotoxins include proteins identified as cry1 through cry43, cyt1 and cyt2, and Cyt-like toxin. There are currently over 250 known species of delta-endotoxins with a wide range of specificities and toxicities. For an expansive list see Crickmore et al. (1998), Microbiol. Mol. Biol. Rev. 62:807-813, and for regular updates see Crickmore et al. (2003) “Bacillus thuringiensis toxin nomenclature,” at www.biols.susx.ac.uk/Home/Neil_Crickmore/Bt/index.

Thus, provided herein are novel isolated nucleotide sequences that confer pesticidal activity. These isolated nucleotide sequences encode polypeptides with homology to known delta-endotoxins or binary toxins. Also provided are the amino acid sequences of the pesticidal proteins. The protein resulting from translation of this gene allows cells to control or kill pests that ingest it.

### Isolated Nucleic Acid Molecules, and Variants and Fragments Thereof

One aspect of the invention pertains to isolated or recombinant nucleic acid molecules comprising nucleotide sequences encoding pesticidal proteins and polypeptides or biologically active portions thereof, as well as nucleic acid molecules sufficient for use as hybridization probes to identify nucleic acid molecules encoding proteins with regions of sequence homology. As used herein, the term “nucleic acid molecule” is intended to include DNA molecules (e.g., recombinant DNA, cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” or “purified” nucleic acid molecule or protein, or biologically active portion thereof, is substantially free of other cellular material, or culture medium when produced by recombinant techniques, or substantially free of chemical precursors or other chemicals when chemically synthesized. Preferably, an “isolated” nucleic acid is free of sequences (preferably protein encoding sequences) that naturally flank the nucleic acid (i.e., sequences located at the 5′ and 3′ ends of the nucleic acid) in the genomic DNA of the organism from which the nucleic acid is derived. For purposes of the invention, “isolated” when used to refer to nucleic acid molecules excludes isolated chromosomes. For example, in various embodiments, the isolated nucleic acid molecule encoding a pesticidal protein can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A pesticidal protein that is substantially free of cellular material includes preparations of protein having less than about 30%, 20%, 10%, or 5% (by dry weight) of non-pesticidal protein (also referred to herein as a “contaminating protein”).

Nucleotide sequences encoding the proteins of the present invention include the sequence set forth in SEQ ID NO:1, 3, 4, 6, 9, or 11, or the nucleotide sequence deposited in a bacterial host as Accession No. B-50045, and variants, fragments, and complements thereof. By “complement” is intended a nucleotide sequence that is sufficiently complementary to a given nucleotide sequence such that it can hybridize to the given nucleotide sequence to thereby form a stable duplex. The corresponding amino acid sequence for the pesticidal protein encoded by this nucleotide sequence are set forth in SEQ ID NO:2 or 5.

Nucleic acid molecules that are fragments of these nucleotide sequences encoding pesticidal proteins are also encompassed by the present invention. By “fragment” is intended a portion of the nucleotide sequence encoding a pesticidal protein. A fragment of a nucleotide sequence may encode a biologically active portion of a pesticidal protein, or it may be a fragment that can be used as a hybridization probe or PCR primer using methods disclosed below. Nucleic acid molecules that are fragments of a nucleotide sequence encoding a pesticidal protein comprise at least about 50, 100, 200, 300, 400, 500, 600, 700, 800, 900, 1000, 1100, 1200, 1300, 1350, 1400 contiguous nucleotides, or up to the number of nucleotides present in a full-length nucleotide sequence encoding a pesticidal protein disclosed herein, depending upon the intended use. By “contiguous” nucleotides is intended nucleotide residues that are immediately adjacent to one another. Fragments of the nucleotide sequences of the present invention will encode protein fragments that retain the biological activity of the pesticidal protein and, hence, retain pesticidal activity. By “retains activity” is intended that the fragment will have at least about 30%, at least about 50%, at least about 70%, 80%, 90%, 95% or higher of the pesticidal activity of the pesticidal protein. In one embodiment, the pesticidal activity is coleoptericidal activity. In another embodiment, the pesticidal activity is lepidoptericidal activity. In another embodiment, the pesticidal activity is nematocidal activity. In another embodiment, the pesticidal activity is diptericidal activity. Methods for measuring pesticidal activity are well known in the art. See, for example, Czapla and Lang (1990) J. Econ. Entomol. 83:2480-2485; Andrews et al. (1988) Biochem. J. 252:199-206; Marrone et al. (1985) J. of Economic Entomology 78:290-293; and U.S. Pat. No. 5,743,477, all of which are herein incorporated by reference in their entirety.

A fragment of a nucleotide sequence encoding a pesticidal protein that encodes a biologically active portion of a protein of the invention will encode at least about 15, 25, 30, 50, 75, 100, 125, 150, 175, 200, 250, 300, 350, 400, 450 contiguous amino acids, or up to the total number of amino acids present in a full-length pesticidal protein of the invention.

Preferred pesticidal proteins of the present invention are encoded by a nucleotide sequence sufficiently identical to the nucleotide sequence of SEQ ID NO:1, 3, 4, 6, 9, or 11. By “sufficiently identical” is intended an amino acid or nucleotide sequence that has at least about 60% or 65% sequence identity, about 70% or 75% sequence identity, about 80% or 85% sequence identity, about 90%, 91%, 92%, 93%, 94%, 95%, 96%, 97%, 98%, 99% or greater sequence identity compared to a reference sequence using one of the alignment programs described herein using standard parameters. One of skill in the art will recognize that these values can be appropriately adjusted to determine corresponding identity of proteins encoded by two nucleotide sequences by taking into account codon degeneracy, amino acid similarity, reading frame positioning, and the like.

To determine the percent identity of two amino acid sequences or of two nucleic acids, the sequences are aligned for optimal comparison purposes. The percent identity between the two sequences is a function of the number of identical positions shared by the sequences (i.e., percent identity=number of identical positions/total number of positions (e.g., overlapping positions)×100). In one embodiment, the two sequences are the same length. In another embodiment, the percent identity is calculated across the entirety of the reference sequence (i.e., the sequence disclosed herein as any of SEQ ID NO:1-13). The percent identity between two sequences can be determined using techniques similar to those described below, with or without allowing gaps. In calculating percent identity, typically exact matches are counted.

The determination of percent identity between two sequences can be accomplished using a mathematical algorithm. A nonlimiting example of a mathematical algorithm utilized for the comparison of two sequences is the algorithm of Karlin and Altschul (1990) Proc. Natl. Acad. Sci. USA 87:2264, modified as in Karlin and Altschul (1993) Proc. Natl. Acad. Sci. USA 90:5873-5877. Such an algorithm is incorporated into the BLASTN and BLASTX programs of Altschul et al. (1990) J. Mol. Biol. 215:403. BLAST nucleotide searches can be performed with the BLASTN program, score=100, wordlength=12, to obtain nucleotide sequences homologous to pesticidal-like nucleic acid molecules of the invention. BLAST protein searches can be performed with the BLASTX program, score=50, wordlength=3, to obtain amino acid sequences homologous to pesticidal protein molecules of the invention. To obtain gapped alignments for comparison purposes, Gapped BLAST (in BLAST 2.0) can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g., BLASTX and BLASTN) can be used. Alignment may also be performed manually by inspection.

Another non-limiting example of a mathematical algorithm utilized for the comparison of sequences is the ClustalW algorithm (Higgins et al. (1994) Nucleic Acids Res. 22:4673-4680). ClustalW compares sequences and aligns the entirety of the amino acid or DNA sequence, and thus can provide data about the sequence conservation of the entire amino acid sequence. The ClustalW algorithm is used in several commercially available DNA/amino acid analysis software packages, such as the ALIGNX module of the Vector NTI Program Suite (Invitrogen Corporation, Carlsbad, Calif.). After alignment of amino acid sequences with ClustalW, the percent amino acid identity can be assessed. A non-limiting example of a software program useful for analysis of ClustalW alignments is GENEDOC™. GENEDOC™ (Karl Nicholas) allows assessment of amino acid (or DNA) similarity and identity between multiple proteins. Another non-limiting example of a mathematical algorithm utilized for the comparison of sequences is the algorithm of Myers and Miller (1988) CABIOS 4: 11-17. Such an algorithm is incorporated into the ALIGN program (version 2.0), which is part of the GCG Wisconsin Genetics Software Package, Version 10 (available from Accelrys, Inc., 9685 Scranton Rd., San Diego, Calif., USA). When utilizing the ALIGN program for comparing amino acid sequences, a PAM120 weight residue table, a gap length penalty of 12, and a gap penalty of 4 can be used.

Unless otherwise stated, GAP Version 10, which uses the algorithm of Needleman and Wunsch (1970) J. Mol. Biol. 48(3):443-453, will be used to determine sequence identity or similarity using the following parameters: % identity and % similarity for a nucleotide sequence using GAP Weight of 50 and Length Weight of 3, and the nwsgapdna.cmp scoring matrix; % identity or % similarity for an amino acid sequence using GAP weight of 8 and length weight of 2, and the BLOSUM62 scoring program. Equivalent programs may also be used. By “equivalent program” is intended any sequence comparison program that, for any two sequences in question, generates an alignment having identical nucleotide residue matches and an identical percent sequence identity when compared to the corresponding alignment generated by GAP Version 10.

The invention also encompasses variant nucleic acid molecules. “Variants” of the pesticidal protein encoding nucleotide sequences include those sequences that encode the pesticidal proteins disclosed herein but that differ conservatively because of the degeneracy of the genetic code as well as those that are sufficiently identical as discussed above. Naturally occurring allelic variants can be identified with the use of well-known molecular biology techniques, such as polymerase chain reaction (PCR) and hybridization techniques as outlined below. Variant nucleotide sequences also include synthetically derived nucleotide sequences that have been generated, for example, by using site-directed mutagenesis but which still encode the pesticidal proteins disclosed in the present invention as discussed below. Variant proteins encompassed by the present invention are biologically active, that is they continue to possess the desired biological activity of the native protein, that is, pesticidal activity. By “retains activity” is intended that the variant will have at least about 30%, at least about 50%, at least about 70%, or at least about 80% of the pesticidal activity of the native protein. Methods for measuring pesticidal activity are well known in the art. See, for example, Czapla and Lang (1990) J. Econ. Entomol. 83: 2480-2485; Andrews et al. (1988) Biochem. J. 252:199-206; Marrone et al. (1985) J. of Economic Entomology 78:290-293; and U.S. Pat. No. 5,743,477, all of which are herein incorporated by reference in their entirety.

In one embodiment, the variants encompass insertion of one or more amino acids into SEQ ID NO:2, 5, or 10. In another embodiment, the variants encompass insertion of one or more amino acids in apical loop 2 of SEQ ID NO:10. In another embodiment, the variants encompass insertion of one or more amino acids between residues 379 and 380 of SEQ ID NO:10. In another embodiment, the variants encompass insertion of at least a glycine residue between residues 379 and 380 of SEQ ID NO:10. In another embodiment, the variants encompass insertion of a glycine residue and one additional residue between residues 379 and 380 of SEQ ID NO:10. In another embodiment, the variants encompass insertion of two glycine residues, of a glycine and a threonine, a glycine and a serine, a glycine and a leucine, an arginine and a glycine, a glycine and an asparagine, a glycine and a lysine, a histidine and a glycine, a phenylalanine and a glycine, a leucine and a glycine, or an asparagine and a glycine residue between residues 379 and 380 of SEQ ID NO:10.

In yet another embodiment, the variant is selected from the group consisting of P83T, L250I, G319K, G319F, I322S, I322V, I322Q, I322A, L323F, Y376N, Y376I, Y376R, Y376S, Y376V, Y376A, R377E, R377Q, R377L, G378S, G378A, G378W, D379V, D379E, L380M, L380P, L380Y, Q381L, L401I, M406H, M406V, M406K, M406E, M406T, M406S, M406A, M406V, M406N, F407W, and F407R relative to SEQ ID NO:10.

The skilled artisan will further appreciate that changes can be introduced by mutation of the nucleotide sequences of the invention thereby leading to changes in the amino acid sequence of the encoded pesticidal proteins, without altering the biological activity of the proteins. Thus, variant isolated nucleic acid molecules can be created by introducing one or more nucleotide substitutions, additions, or deletions into the corresponding nucleotide sequence disclosed herein, such that one or more amino acid substitutions, additions or deletions are introduced into the encoded protein. Mutations can be introduced by standard techniques, such as site-directed mutagenesis and PCR-mediated mutagenesis. Such variant nucleotide sequences are also encompassed by the present invention.

For example, conservative amino acid substitutions may be made at one or more, predicted, nonessential amino acid residues. A “nonessential” amino acid residue is a residue that can be altered from the wild-type sequence of a pesticidal protein without altering the biological activity, whereas an “essential” amino acid residue is required for biological activity. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Delta-endotoxins generally have five conserved sequence domains, and three conserved structural domains (see, for example, de Maagd et al. (2001) Trends Genetics 17:193-199). The first conserved structural domain consists of seven alpha helices and is involved in membrane insertion and pore formation. Domain II consists of three beta-sheets arranged in a Greek key configuration, and domain III consists of two antiparallel beta-sheets in “jelly-roll” formation (de Maagd et al., 2001, supra). Domains II and III are involved in receptor recognition and binding, and are therefore considered determinants of toxin specificity.

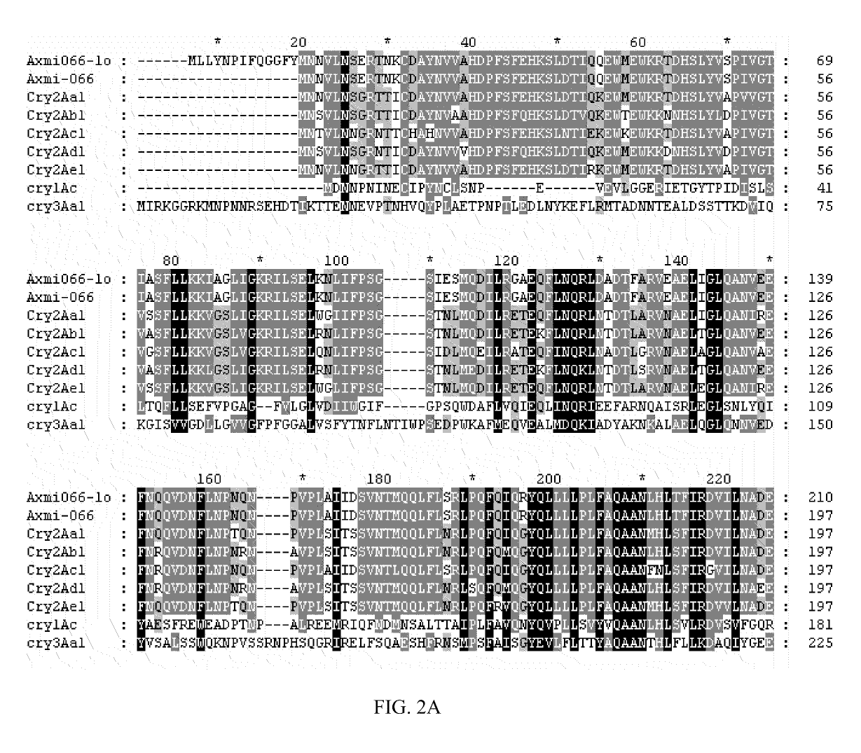

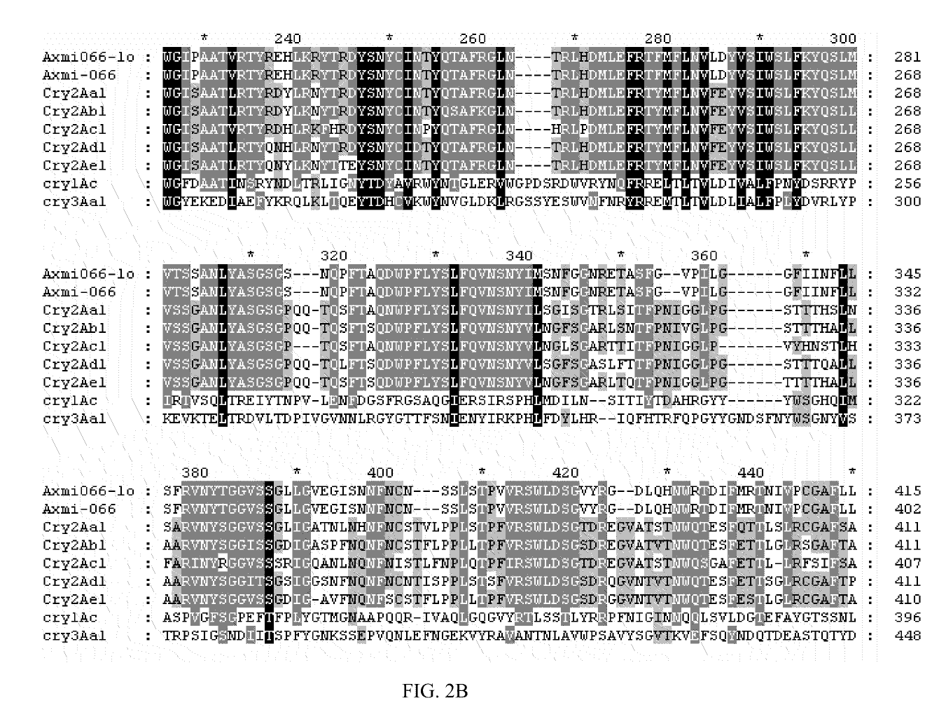

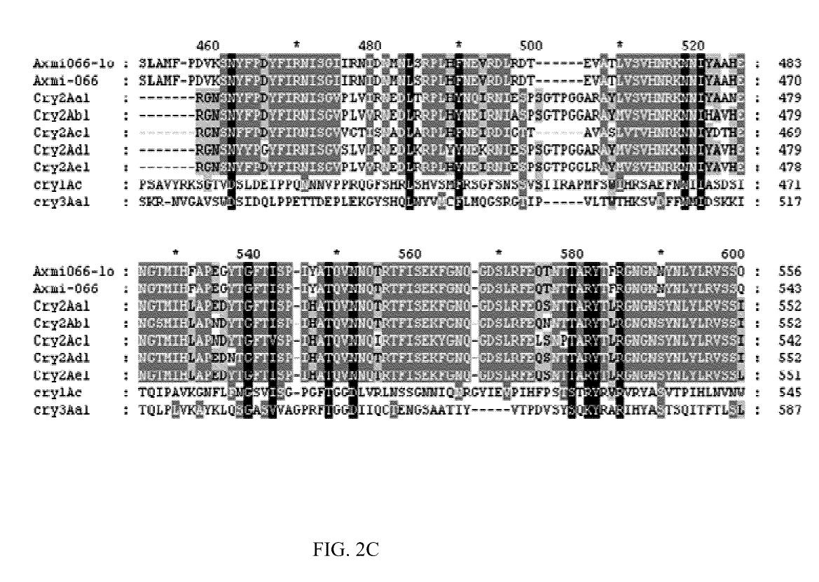

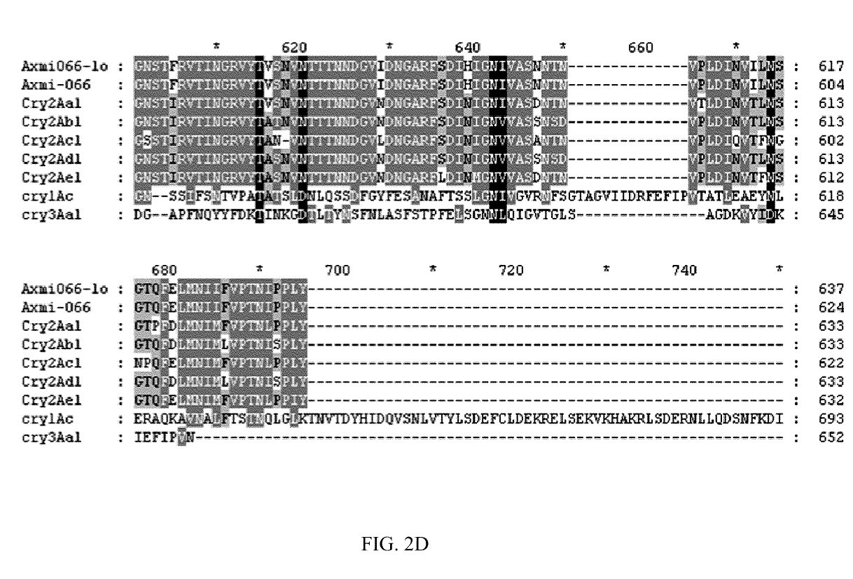

AXMI-066 shows homology Cry2A family of proteins. The 3D structure of Cry2Aa has been determined (see, Morse et al. (2001) Structure 9:409-417), and domain swapping experiments between Cry2A and Cry2B have lead to the identification of specificity regions (see, for example, Liang and Dean (1994) Molecular Microbiology, 13 (4):569-575; Widner and Whiteley (1989) J. Bacteriology. 171(2)965-974; and Widner and Whiteley (1990) J. Bacteriology., 172(6):2826-2832, each of which is herein incorporated by reference in its entirety). Apical loops in Cry toxins have been implicated in receptor recognition, and Cry2Aa contains 2 apical loops. Loop 1 is found from about position 316 to about position 335 of SEQ ID NO:14. Loop 2 is found from about position 370 to about position 394 of SEQ ID NO:14. The corresponding residues in AXMI-066 can be found in the alignment of FIG. 2.

Amino acid substitutions may be made in nonconserved regions that retain function. In general, such substitutions would not be made for conserved amino acid residues, or for amino acid residues residing within a conserved motif, where such residues are essential for protein activity. Examples of residues that are conserved and that may be essential for protein activity include, for example, residues that are identical between all proteins contained in an alignment of similar or related toxins to the sequences of the invention (e.g., residues that are identical between all proteins contained in the alignment in FIG. 7). Examples of residues that are conserved but that may allow conservative amino acid substitutions and still retain activity include, for example, residues that have only conservative substitutions between all proteins contained in an alignment of similar or related toxins to the sequences of the invention (e.g., residues that have only conservative substitutions between all proteins contained in the alignment in FIG. 7). However, one of skill in the art would understand that functional variants may have minor conserved or nonconserved alterations in the conserved residues.

Alternatively, variant nucleotide sequences can be made by introducing mutations randomly along all or part of the coding sequence, such as by saturation mutagenesis, and the resultant mutants can be screened for ability to confer pesticidal activity to identify mutants that retain activity. Following mutagenesis, the encoded protein can be expressed recombinantly, and the activity of the protein can be determined using standard assay techniques.

Using methods such as PCR, hybridization, and the like corresponding pesticidal sequences can be identified, such sequences having substantial identity to the sequences of the invention. See, for example, Sambrook and Russell (2001) Molecular Cloning: A Laboratory Manual. (Cold Spring Harbor Laboratory Press, Cold Spring Harbor, N.Y.) and Innis, et al. (1990) PCR Protocols: A Guide to Methods and Applications (Academic Press, NY).

In a hybridization method, all or part of the pesticidal nucleotide sequence can be used to screen cDNA or genomic libraries. Methods for construction of such cDNA and genomic libraries are generally known in the art and are disclosed in Sambrook and Russell, 2001, supra. The so-called hybridization probes may be genomic DNA fragments, cDNA fragments, RNA fragments, or other oligonucleotides, and may be labeled with a detectable group such as ³²P, or any other detectable marker, such as other radioisotopes, a fluorescent compound, an enzyme, or an enzyme co-factor. Probes for hybridization can be made by labeling synthetic oligonucleotides based on the known pesticidal protein-encoding nucleotide sequence disclosed herein. Degenerate primers designed on the basis of conserved nucleotides or amino acid residues in the nucleotide sequence or encoded amino acid sequence can additionally be used. The probe typically comprises a region of nucleotide sequence that hybridizes under stringent conditions to at least about 12, at least about 25, at least about 50, 75, 100, 125, 150, 175, or 200 consecutive nucleotides of nucleotide sequence encoding a pesticidal protein of the invention or a fragment or variant thereof. Methods for the preparation of probes for hybridization are generally known in the art and are disclosed in Sambrook and Russell, 2001, supra herein incorporated by reference.

For example, an entire pesticidal protein sequence disclosed herein, or one or more portions thereof, may be used as a probe capable of specifically hybridizing to corresponding pesticidal protein-like sequences and messenger RNAs. To achieve specific hybridization under a variety of conditions, such probes include sequences that are unique and are preferably at least about 10 nucleotides in length, or at least about 20 nucleotides in length. Such probes may be used to amplify corresponding pesticidal sequences from a chosen organism by PCR. This technique may be used to isolate additional coding sequences from a desired organism or as a diagnostic assay to determine the presence of coding sequences in an organism. Hybridization techniques include hybridization screening of plated DNA libraries (either plaques or colonies; see, for example, Sambrook et al. (1989) Molecular Cloning: A Laboratory Manual (2d ed., Cold Spring Harbor Laboratory Press, Cold Spring Harbor, N.Y.).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. Optionally, wash buffers may comprise about 0.1% to about 1% SDS. Duration of hybridization is generally less than about 24 hours, usually about 4 to about 12 hours.

Specificity is typically the function of post-hybridization washes, the critical factors being the ionic strength and temperature of the final wash solution. For DNA-DNA hybrids, the Tₘ can be approximated from the equation of Meinkoth and Wahl (1984) Anal. Biochem. 138:267-284: Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is preferred to increase the SSC concentration so that a higher temperature can be used. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, New York); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Cold Spring Harbor, N.Y.).

### Isolated Proteins and Variants and Fragments Thereof

Pesticidal proteins are also encompassed within the present invention. By “pesticidal protein” is intended a protein having the amino acid sequence set forth in SEQ ID NO:2 or 5. Fragments, biologically active portions, and variants thereof are also provided, and may be used to practice the methods of the present invention.

“Fragments” or “biologically active portions” include polypeptide fragments comprising amino acid sequences sufficiently identical to the amino acid sequence set forth in SEQ ID NO:2 or 5, and that exhibit pesticidal activity. A biologically active portion of a pesticidal protein can be a polypeptide that is, for example, 10, 25, 50, 100, 150, 200, 250 or more amino acids in length. Such biologically active portions can be prepared by recombinant techniques and evaluated for pesticidal activity. Methods for measuring pesticidal activity are well known in the art. See, for example, Czapla and Lang (1990) J. Econ. Entomol. 83:2480-2485; Andrews et al. (1988) Biochem. J. 252:199-206; Marrone et al. (1985) J. of Economic Entomology 78:290-293; and U.S. Pat. No. 5,743,477, all of which are herein incorporated by reference in their entirety. As used here, a fragment comprises at least 8 contiguous amino acids of SEQ ID NO:2 or 5. The invention encompasses other fragments, however, such as any fragment in the protein greater than about 10, 20, 30, 50, 100, 150, 200, 250, or 300 amino acids.

By “variants” is intended proteins or polypeptides having an amino acid sequence that is at least about 60%, 65%, about 70%, 75%, about 80%, 85%, about 90%, 91%, 92%, 93%, 94%, 95%, 96%, 97%, 98% or 99% identical to the amino acid sequence of SEQ ID NO:2 or 5. Variants also include polypeptides encoded by a nucleic acid molecule that hybridizes to the nucleic acid molecule of SEQ ID NO:1, 3, 4, 6, 9, or 11, or a complement thereof, under stringent conditions. Variants include polypeptides that differ in amino acid sequence due to mutagenesis. Variant proteins encompassed by the present invention are biologically active, that is they continue to possess the desired biological activity of the native protein, that is, retaining pesticidal activity. Methods for measuring pesticidal activity are well known in the art. See, for example, Czapla and Lang (1990) J. Econ. Entomol. 83:2480-2485; Andrews et al. (1988) Biochem. J. 252:199-206; Marrone et al. (1985) J. of Economic Entomology 78:290-293; and U.S. Pat. No. 5,743,477, all of which are herein incorporated by reference in their entirety.

Bacterial genes, such as the axmi genes of this invention, quite often possess multiple methionine initiation codons in proximity to the start of the open reading frame. Often, translation initiation at one or more of these start codons will lead to generation of a functional protein. These start codons can include ATG codons. However, bacteria such as Bacillus sp. also recognize the codon GTG as a start codon, and proteins that initiate translation at GTG codons contain a methionine at the first amino acid. Furthermore, it is not often determined a priori which of these codons are used naturally in the bacterium. Thus, it is understood that use of one of the alternate methionine codons may also lead to generation of pesticidal proteins. These pesticidal proteins are encompassed in the present invention and may be used in the methods of the present invention.

Antibodies to the polypeptides of the present invention, or to variants or fragments thereof, are also encompassed. Methods for producing antibodies are well known in the art (see, for example, Harlow and Lane (1988) Antibodies: A Laboratory Manual, Cold Spring Harbor Laboratory, Cold Spring Harbor, N.Y.; U.S. Pat. No. 4,196,265).

### Altered or Improved Variants

It is recognized that DNA sequences of a pesticidal protein may be altered by various methods, and that these alterations may result in DNA sequences encoding proteins with amino acid sequences different than that encoded by a pesticidal protein of the present invention. This protein may be altered in various ways including amino acid substitutions, deletions, truncations, and insertions of one or more amino acids of SEQ ID NO:2 or 5, including up to about 2, about 3, about 4, about 5, about 6, about 7, about 8, about 9, about 10, about 15, about 20, about 25, about 30, about 35, about 40, about 45, about 50, about 55, about 60, about 65, about 70, about 75, about 80, about 85, about 90, about 100, about 105, about 110, about 115, about 120, about 125, about 130, about 135, about 140, about 145, about 150, about 155, or more amino acid substitutions, deletions or insertions. Methods for such manipulations are generally known in the art. For example, amino acid sequence variants of a pesticidal protein can be prepared by mutations in the DNA. This may also be accomplished by one of several forms of mutagenesis and/or in directed evolution. In some aspects, the changes encoded in the amino acid sequence will not substantially affect the function of the protein. Such variants will possess the desired pesticidal activity. However, it is understood that the ability of a pesticidal protein to confer pesticidal activity may be improved by the use of such techniques upon the compositions of this invention. For example, one may express a pesticidal protein in host cells that exhibit high rates of base misincorporation during DNA replication, such as XL-1 Red (Stratagene, La Jolla, Calif.). After propagation in such strains, one can isolate the DNA (for example by preparing plasmid DNA, or by amplifying by PCR and cloning the resulting PCR fragment into a vector), culture the pesticidal protein mutations in a non-mutagenic strain, and identify mutated genes with pesticidal activity, for example by performing an assay to test for pesticidal activity. Generally, the protein is mixed and used in feeding assays. See, for example Marrone et al. (1985) J. of Economic Entomology 78:290-293. Such assays can include contacting plants with one or more pests and determining the plant's ability to survive and/or cause the death of the pests. Examples of mutations that result in increased toxicity are found in Schnepf et al. (1998) Microbiol. Mol. Biol. Rev. 62:775-806.

Alternatively, alterations may be made to the protein sequence of many proteins at the amino or carboxy terminus without substantially affecting activity. This can include insertions, deletions, or alterations introduced by modern molecular methods, such as PCR, including PCR amplifications that alter or extend the protein coding sequence by virtue of inclusion of amino acid encoding sequences in the oligonucleotides utilized in the PCR amplification. Alternatively, the protein sequences added can include entire protein-coding sequences, such as those used commonly in the art to generate protein fusions. Such fusion proteins are often used to (1) increase expression of a protein of interest (2) introduce a binding domain, enzymatic activity, or epitope to facilitate either protein purification, protein detection, or other experimental uses known in the art (3) target secretion or translation of a protein to a subcellular organelle, such as the periplasmic space of Gram-negative bacteria, or the endoplasmic reticulum of eukaryotic cells, the latter of which often results in glycosylation of the protein.

Variant nucleotide and amino acid sequences of the present invention also encompass sequences derived from mutagenic and recombinogenic procedures such as DNA shuffling. With such a procedure, one or more different pesticidal protein coding regions can be used to create a new pesticidal protein possessing the desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between a pesticidal gene of the invention and other known pesticidal genes to obtain a new gene coding for a protein with an improved property of interest, such as an increased insecticidal activity. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458.

Domain swapping or shuffling is another mechanism for generating altered pesticidal proteins. Domains may be swapped between pesticidal proteins, resulting in hybrid or chimeric toxins with improved pesticidal activity or target spectrum. Methods for generating recombinant proteins and testing them for pesticidal activity are well known in the art (see, for example, Naimov et al. (2001) Appl. Environ. Microbiol. 67:5328-5330; de Maagd et al. (1996) Appl. Environ. Microbiol. 62:1537-1543; Ge et al. (1991) J. Biol. Chem. 266:17954-17958; Schnepf et al. (1990) J. Biol. Chem. 265:20923-20930; Rang et al. 91999) Appl. Environ. Microbiol. 65:2918-2925).

### Vectors

A pesticidal sequence of the invention may be provided in an expression cassette for expression in a plant of interest. By “plant expression cassette” is intended a DNA construct that is capable of resulting in the expression of a protein from an open reading frame in a plant cell. Typically these contain a promoter and a coding sequence. Often, such constructs will also contain a 3′ untranslated region. Such constructs may contain a “signal sequence” or “leader sequence” to facilitate co-translational or post-translational transport of the peptide to certain intracellular structures such as the chloroplast (or other plastid), endoplasmic reticulum, or Golgi apparatus.

By “signal sequence” is intended a sequence that is known or suspected to result in cotranslational or post-translational peptide transport across the cell membrane. In eukaryotes, this typically involves secretion into the Golgi apparatus, with some resulting glycosylation. Insecticidal toxins of bacteria are often synthesized as protoxins, which are protolytically activated in the gut of the target pest (Chang (1987) Methods Enzymol. 153:507-516). In some embodiments of the present invention, the signal sequence is located in the native sequence, or may be derived from a sequence of the invention. By “leader sequence” is intended any sequence that when translated, results in an amino acid sequence sufficient to trigger co-translational transport of the peptide chain to a subcellular organelle. Thus, this includes leader sequences targeting transport and/or glycosylation by passage into the endoplasmic reticulum, passage to vacuoles, plastids including chloroplasts, mitochondria, and the like.

By “plant transformation vector” is intended a DNA molecule that is necessary for efficient transformation of a plant cell. Such a molecule may consist of one or more plant expression cassettes, and may be organized into more than one “vector” DNA molecule. For example, binary vectors are plant transformation vectors that utilize two non-contiguous DNA vectors to encode all requisite cis- and trans-acting functions for transformation of plant cells (Hellens and Mullineaux (2000) Trends in Plant Science 5:446-451). “Vector” refers to a nucleic acid construct designed for transfer between different host cells. “Expression vector” refers to a vector that has the ability to incorporate, integrate and express heterologous DNA sequences or fragments in a foreign cell. The cassette will include 5′ and 3′ regulatory sequences operably linked to a sequence of the invention. By “operably linked” is intended a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame. The cassette may additionally contain at least one additional gene to be cotransformed into the organism. Alternatively, the additional gene(s) can be provided on multiple expression cassettes.

“Promoter” refers to a nucleic acid sequence that functions to direct transcription of a downstream coding sequence. The promoter together with other transcriptional and translational regulatory nucleic acid sequences (also termed “control sequences”) are necessary for the expression of a DNA sequence of interest.

Such an expression cassette is provided with a plurality of restriction sites for insertion of the pesticidal sequence to be under the transcriptional regulation of the regulatory regions.

The expression cassette will include in the 5′-3′ direction of transcription, a transcriptional and translational initiation region (i.e., a promoter), a DNA sequence of the invention, and a translational and transcriptional termination region (i.e., termination region) functional in plants. The promoter may be native or analogous, or foreign or heterologous, to the plant host and/or to the DNA sequence of the invention. Additionally, the promoter may be the natural sequence or alternatively a synthetic sequence. Where the promoter is “native” or “homologous” to the plant host, it is intended that the promoter is found in the native plant into which the promoter is introduced. Where the promoter is “foreign” or “heterologous” to the DNA sequence of the invention, it is intended that the promoter is not the native or naturally occurring promoter for the operably linked DNA sequence of the invention.

The termination region may be native with the transcriptional initiation region, may be native with the operably linked DNA sequence of interest, may be native with the plant host, or may be derived from another source (i.e., foreign or heterologous to the promoter, the DNA sequence of interest, the plant host, or any combination thereof). Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acid Res. 15:9627-9639.

Where appropriate, the gene(s) may be optimized for increased expression in the transformed host cell. That is, the genes can be synthesized using host cell-preferred codons for improved expression, or may be synthesized using codons at a host-preferred codon usage frequency. Generally, the GC content of the gene will be increased. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

In one embodiment, the pesticidal protein is targeted to the chloroplast for expression. In this manner, where the pesticidal protein is not directly inserted into the chloroplast, the expression cassette will additionally contain a nucleic acid encoding a transit peptide to direct the pesticidal protein to the chloroplasts. Such transit peptides are known in the art. See, for example, Von Heijne et al. (1991) Plant Mol. Biol. Rep. 9:104-126; Clark et al. (1989) J. Biol. Chem. 264:17544-17550; Della-Cioppa et al. (1987) Plant Physiol. 84:965-968; Romer et al. (1993) Biochem. Biophys. Res. Commun. 196:1414-1421; and Shah et al. (1986) Science 233:478-481.

The pesticidal gene to be targeted to the chloroplast may be optimized for expression in the chloroplast to account for differences in codon usage between the plant nucleus and this organelle. In this manner, the nucleic acids of interest may be synthesized using chloroplast-preferred codons. See, for example, U.S. Pat. No. 5,380,831, herein incorporated by reference.

### Plant Transformation

Methods of the invention involve introducing a nucleotide construct into a plant. By “introducing” is intended to present to the plant the nucleotide construct in such a manner that the construct gains access to the interior of a cell of the plant. The methods of the invention do not require that a particular method for introducing a nucleotide construct to a plant is used, only that the nucleotide construct gains access to the interior of at least one cell of the plant. Methods for introducing nucleotide constructs into plants are known in the art including, but not limited to, stable transformation methods, transient transformation methods, and virus-mediated methods.

By “plant” is intended whole plants, plant organs (e.g., leaves, stems, roots, etc.), seeds, plant cells, propagules, embryos and progeny of the same. Plant cells can be differentiated or undifferentiated (e.g. callus, suspension culture cells, protoplasts, leaf cells, root cells, phloem cells, pollen).

“Transgenic plants” or “transformed plants” or “stably transformed” plants or cells or tissues refers to plants that have incorporated or integrated exogenous nucleic acid sequences or DNA fragments into the plant cell. These nucleic acid sequences include those that are exogenous, or not present in the untransformed plant cell, as well as those that may be endogenous, or present in the untransformed plant cell. “Heterologous” generally refers to the nucleic acid sequences that are not endogenous to the cell or part of the native genome in which they are present, and have been added to the cell by infection, transfection, microinjection, electroporation, microprojection, or the like.

Transformation of plant cells can be accomplished by one of several techniques known in the art. The pesticidal gene of the invention may be modified to obtain or enhance expression in plant cells. Typically a construct that expresses such a protein would contain a promoter to drive transcription of the gene, as well as a 3′ untranslated region to allow transcription termination and polyadenylation. The organization of such constructs is well known in the art. In some instances, it may be useful to engineer the gene such that the resulting peptide is secreted, or otherwise targeted within the plant cell. For example, the gene can be engineered to contain a signal peptide to facilitate transfer of the peptide to the endoplasmic reticulum. It may also be preferable to engineer the plant expression cassette to contain an intron, such that mRNA processing of the intron is required for expression.

Typically this “plant expression cassette” will be inserted into a “plant transformation vector”. This plant transformation vector may be comprised of one or more DNA vectors needed for achieving plant transformation. For example, it is a common practice in the art to utilize plant transformation vectors that are comprised of more than one contiguous DNA segment. These vectors are often referred to in the art as “binary vectors”. Binary vectors as well as vectors with helper plasmids are most often used for Agrobacterium-mediated transformation, where the size and complexity of DNA segments needed to achieve efficient transformation is quite large, and it is advantageous to separate functions onto separate DNA molecules. Binary vectors typically contain a plasmid vector that contains the cis-acting sequences required for T-DNA transfer (such as left border and right border), a selectable marker that is engineered to be capable of expression in a plant cell, and a “gene of interest” (a gene engineered to be capable of expression in a plant cell for which generation of transgenic plants is desired). Also present on this plasmid vector are sequences required for bacterial replication. The cis-acting sequences are arranged in a fashion to allow efficient transfer into plant cells and expression therein. For example, the selectable marker gene and the pesticidal gene are located between the left and right borders. Often a second plasmid vector contains the trans-acting factors that mediate T-DNA transfer from Agrobacterium to plant cells. This plasmid often contains the virulence functions (Vir genes) that allow infection of plant cells by Agrobacterium, and transfer of DNA by cleavage at border sequences and vir-mediated DNA transfer, as is understood in the art (Hellens and Mullineaux (2000) Trends in Plant Science 5:446-451). Several types of Agrobacterium strains (e.g. LBA4404, GV3101, EHA101, EHA105, etc.) can be used for plant transformation. The second plasmid vector is not necessary for transforming the plants by other methods such as microprojection, microinjection, electroporation, polyethylene glycol, etc.

In general, plant transformation methods involve transferring heterologous DNA into target plant cells (e.g. immature or mature embryos, suspension cultures, undifferentiated callus, protoplasts, etc.), followed by applying a maximum threshold level of appropriate selection (depending on the selectable marker gene) to recover the transformed plant cells from a group of untransformed cell mass. Explants are typically transferred to a fresh supply of the same medium and cultured routinely. Subsequently, the transformed cells are differentiated into shoots after placing on regeneration medium supplemented with a maximum threshold level of selecting agent. The shoots are then transferred to a selective rooting medium for recovering rooted shoot or plantlet. The transgenic plantlet then grows into a mature plant and produces fertile seeds (e.g. Hiei et al. (1994) The Plant Journal 6:271-282; Ishida et al. (1996) Nature Biotechnology 14:745-750). Explants are typically transferred to a fresh supply of the same medium and cultured routinely. A general description of the techniques and methods for generating transgenic plants are found in Ayres and Park (1994) Critical Reviews in Plant Science 13:219-239 and Bommineni and Jauhar (1997) Maydica 42:107-120. Since the transformed material contains many cells; both transformed and non-transformed cells are present in any piece of subjected target callus or tissue or group of cells. The ability to kill non-transformed cells and allow transformed cells to proliferate results in transformed plant cultures. Often, the ability to remove non-transformed cells is a limitation to rapid recovery of transformed plant cells and successful generation of transgenic plants.

Transformation protocols as well as protocols for introducing nucleotide sequences into plants may vary depending on the type of plant or plant cell, i.e., monocot or dicot, targeted for transformation. Generation of transgenic plants may be performed by one of several methods, including, but not limited to, microinjection, electroporation, direct gene transfer, introduction of heterologous DNA by Agrobacterium into plant cells (Agrobacterium-mediated transformation), bombardment of plant cells with heterologous foreign DNA adhered to particles, ballistic particle acceleration, aerosol beam transformation (U.S. Published Application No. 20010026941; U.S. Pat. No. 4,945,050; International Publication No. WO 91/00915; U.S. Published Application No. 2002015066), Lec1 transformation, and various other non-particle direct-mediated methods to transfer DNA.

Methods for transformation of chloroplasts are known in the art. See, for example, Svab et al. (1990) Proc. Natl. Acad. Sci. USA 87:8526-8530; Svab and Maliga (1993) Proc. Natl. Acad. Sci. USA 90:913-917; Svab and Maliga (1993) EMBO J. 12:601-606. The method relies on particle gun delivery of DNA containing a selectable marker and targeting of the DNA to the plastid genome through homologous recombination. Additionally, plastid transformation can be accomplished by transactivation of a silent plastid-borne transgene by tissue-preferred expression of a nuclear-encoded and plastid-directed RNA polymerase. Such a system has been reported in McBride et al. (1994) Proc. Natl. Acad. Sci. USA 91:7301-7305.

Following integration of heterologous foreign DNA into plant cells, one then applies a maximum threshold level of appropriate selection in the medium to kill the untransformed cells and separate and proliferate the putatively transformed cells that survive from this selection treatment by transferring regularly to a fresh medium. By continuous passage and challenge with appropriate selection, one identifies and proliferates the cells that are transformed with the plasmid vector. Molecular and biochemical methods can then be used to confirm the presence of the integrated heterologous gene of interest into the genome of the transgenic plant.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a nucleotide construct of the invention, for example, an expression cassette of the invention, stably incorporated into their genome.

### Evaluation of Plant Transformation

Following introduction of heterologous foreign DNA into plant cells, the transformation or integration of heterologous gene in the plant genome is confirmed by various methods such as analysis of nucleic acids, proteins and metabolites associated with the integrated gene.

PCR analysis is a rapid method to screen transformed cells, tissue or shoots for the presence of incorporated gene at the earlier stage before transplanting into the soil (Sambrook and Russell (2001) Molecular Cloning: A Laboratory Manual. Cold Spring Harbor Laboratory Press, Cold Spring Harbor, N.Y.). PCR is carried out using oligonucleotide primers specific to the gene of interest or Agrobacterium vector background, etc.

Plant transformation may be confirmed by Southern blot analysis of genomic DNA (Sambrook and Russell, 2001, supra). In general, total DNA is extracted from the transformant, digested with appropriate restriction enzymes, fractionated in an agarose gel and transferred to a nitrocellulose or nylon membrane. The membrane or “blot” is then probed with, for example, radiolabeled ³²P target DNA fragment to confirm the integration of introduced gene into the plant genome according to standard techniques (Sambrook and Russell, 2001, supra).

In Northern blot analysis, RNA is isolated from specific tissues of transformant, fractionated in a formaldehyde agarose gel, and blotted onto a nylon filter according to standard procedures that are routinely used in the art (Sambrook and Russell, 2001, supra). Expression of RNA encoded by the pesticidal gene is then tested by hybridizing the filter to a radioactive probe derived from a pesticidal gene, by methods known in the art (Sambrook and Russell, 2001, supra).

Western blot, biochemical assays and the like may be carried out on the transgenic plants to confirm the presence of protein encoded by the pesticidal gene by standard procedures (Sambrook and Russell, 2001, supra) using antibodies that bind to one or more epitopes present on the pesticidal protein.

### Pesticidal Activity in Plants

In another aspect of the invention, one may generate transgenic plants expressing a pesticidal protein that has pesticidal activity. Methods described above by way of example may be utilized to generate transgenic plants, but the manner in which the transgenic plant cells are generated is not critical to this invention. Methods known or described in the art such as Agrobacterium-mediated transformation, biolistic transformation, and non-particle-mediated methods may be used at the discretion of the experimenter. Plants expressing a pesticidal protein may be isolated by common methods described in the art, for example by transformation of callus, selection of transformed callus, and regeneration of fertile plants from such transgenic callus. In such process, one may use any gene as a selectable marker so long as its expression in plant cells confers ability to identify or select for transformed cells.

A number of markers have been developed for use with plant cells, such as resistance to chloramphenicol, the aminoglycoside G418, hygromycin, or the like. Other genes that encode a product involved in chloroplast metabolism may also be used as selectable markers. For example, genes that provide resistance to plant herbicides such as glyphosate, bromoxynil, or imidazolinone may find particular use. Such genes have been reported (Stalker et al. (1985) J. Biol. Chem. 263:6310-6314 (bromoxynil resistance nitrilase gene); and Sathasivan et al. (1990) Nucl. Acids Res. 18:2188 (AHAS imidazolinone resistance gene). Additionally, the genes disclosed herein are useful as markers to assess transformation of bacterial or plant cells. Methods for detecting the presence of a transgene in a plant, plant organ (e.g., leaves, stems, roots, etc.), seed, plant cell, propagule, embryo or progeny of the same are well known in the art. In one embodiment, the presence of the transgene is detected by testing for pesticidal activity.

Fertile plants expressing a pesticidal protein may be tested for pesticidal activity, and the plants showing optimal activity selected for further breeding. Methods are available in the art to assay for pest activity. Generally, the protein is mixed and used in feeding assays. See, for example Marrone et al. (1985) J. of Economic Entomology 78:290-293.

The present invention may be used for transformation of any plant species, including, but not limited to, monocots and dicots. Examples of plants of interest include, but are not limited to, corn (maize), sorghum, wheat, sunflower, tomato, crucifers, peppers, potato, cotton, rice, soybean, sugarbeet, sugarcane, tobacco, barley, and oilseed rape, Brassica sp., alfalfa, rye, millet, safflower, peanuts, sweet potato, cassava, coffee, coconut, pineapple, citrus trees, cocoa, tea, banana, avocado, fig, guava, mango, olive, papaya, cashew, macadamia, almond, oats, vegetables, ornamentals, and conifers.

Vegetables include, but are not limited to, tomatoes, lettuce, green beans, lima beans, peas, and members of the genus Curcumis such as cucumber, cantaloupe, and musk melon. Ornamentals include, but are not limited to, azalea, hydrangea, hibiscus, roses, tulips, daffodils, petunias, carnation, poinsettia, and chrysanthemum. Preferably, plants of the present invention are crop plants (for example, maize, sorghum, wheat, sunflower, tomato, crucifers, peppers, potato, cotton, rice, soybean, sugarbeet, sugarcane, tobacco, barley, oilseed rape, etc.).

### Use in Pesticidal Control

General methods for employing strains comprising a nucleotide sequence of the present invention, or a variant thereof, in pesticide control or in engineering other organisms as pesticidal agents are known in the art. See, for example U.S. Pat. No. 5,039,523 and EP 0480762A2.

The Bacillus strains containing a nucleotide sequence of the present invention, or a variant thereof, or the microorganisms that have been genetically altered to contain a pesticidal gene and protein may be used for protecting agricultural crops and products from pests. In one aspect of the invention, whole, i.e., unlysed, cells of a toxin (pesticide)-producing organism are treated with reagents that prolong the activity of the toxin produced in the cell when the cell is applied to the environment of target pest(s).

Alternatively, the pesticide is produced by introducing a pesticidal gene into a cellular host. Expression of the pesticidal gene results, directly or indirectly, in the intracellular production and maintenance of the pesticide. In one aspect of this invention, these cells are then treated under conditions that prolong the activity of the toxin produced in the cell when the cell is applied to the environment of target pest(s). The resulting product retains the toxicity of the toxin. These naturally encapsulated pesticides may then be formulated in accordance with conventional techniques for application to the environment hosting a target pest, e.g., soil, water, and foliage of plants. See, for example EPA 0192319, and the references cited therein. Alternatively, one may formulate the cells expressing a gene of this invention such as to allow application of the resulting material as a pesticide.

The active ingredients of the present invention are normally applied in the form of compositions and can be applied to the crop area or plant to be treated, simultaneously or in succession, with other compounds. These compounds can be fertilizers, weed killers, cryoprotectants, surfactants, detergents, pesticidal soaps, dormant oils, polymers, and/or time-release or biodegradable carrier formulations that permit long-term dosing of a target area following a single application of the formulation. They can also be selective herbicides, chemical insecticides, virucides, microbicides, amoebicides, pesticides, fungicides, bacteriocides, nematocides, molluscicides or mixtures of several of these preparations, if desired, together with further agriculturally acceptable carriers, surfactants or application-promoting adjuvants customarily employed in the art of formulation. Suitable carriers and adjuvants can be solid or liquid and correspond to the substances ordinarily employed in formulation technology, e.g. natural or regenerated mineral substances, solvents, dispersants, wetting agents, tackifiers, binders or fertilizers. Likewise the formulations may be prepared into edible “baits” or fashioned into pest “traps” to permit feeding or ingestion by a target pest of the pesticidal formulation.

Methods of applying an active ingredient of the present invention or an agrochemical composition of the present invention that contains at least one of the pesticidal proteins produced by the bacterial strains of the present invention include leaf application, seed coating and soil application. The number of applications and the rate of application depend on the intensity of infestation by the corresponding pest.

The composition may be formulated as a powder, dust, pellet, granule, spray, emulsion, colloid, solution, or such like, and may be prepared by such conventional means as desiccation, lyophilization, homogenation, extraction, filtration, centrifugation, sedimentation, or concentration of a culture of cells comprising the polypeptide. In all such compositions that contain at least one such pesticidal polypeptide, the polypeptide may be present in a concentration of from about 1% to about 99% by weight.

Lepidopteran, dipteran, or coleopteran pests may be killed or reduced in numbers in a given area by the methods of the invention, or may be prophylactically applied to an environmental area to prevent infestation by a susceptible pest. Preferably the pest ingests, or is contacted with, a pesticidally-effective amount of the polypeptide. By “pesticidally-effective amount” is intended an amount of the pesticide that is able to bring about death to at least one pest, or to noticeably reduce pest growth, feeding, or normal physiological development. This amount will vary depending on such factors as, for example, the specific target pests to be controlled, the specific environment, location, plant, crop, or agricultural site to be treated, the environmental conditions, and the method, rate, concentration, stability, and quantity of application of the pesticidally-effective polypeptide composition. The formulations may also vary with respect to climatic conditions, environmental considerations, and/or frequency of application and/or severity of pest infestation.

The pesticide compositions described may be made by formulating either the bacterial cell, crystal and/or spore suspension, or isolated protein component with the desired agriculturally-acceptable carrier. The compositions may be formulated prior to administration in an appropriate means such as lyophilized, freeze-dried, desiccated, or in an aqueous carrier, medium or suitable diluent, such as saline or other buffer. The formulated compositions may be in the form of a dust or granular material, or a suspension in oil (vegetable or mineral), or water or oil/water emulsions, or as a wettable powder, or in combination with any other carrier material suitable for agricultural application. Suitable agricultural carriers can be solid or liquid and are well known in the art. The term “agriculturally-acceptable carrier” covers all adjuvants, inert components, dispersants, surfactants, tackifiers, binders, etc. that are ordinarily used in pesticide formulation technology; these are well known to those skilled in pesticide formulation. The formulations may be mixed with one or more solid or liquid adjuvants and prepared by various means, e.g., by homogeneously mixing, blending and/or grinding the pesticidal composition with suitable adjuvants using conventional formulation techniques. Suitable formulations and application methods are described in U.S. Pat. No. 6,468,523, herein incorporated by reference.

“Pest” includes but is not limited to, insects, fungi, bacteria, nematodes, mites, ticks, and the like. Insect pests include insects selected from the orders Coleoptera, Diptera, Hymenoptera, Lepidoptera, Mallophaga, Homoptera, Hemiptera, Orthroptera, Thysanoptera, Dermaptera, Isoptera, Anoplura, Siphonaptera, Trichoptera, etc., particularly Coleoptera, Lepidoptera, and Diptera.

The order Coleoptera includes the suborders Adephaga and Polyphaga. Suborder Adephaga includes the superfamilies Caraboidea and Gyrinoidea, while suborder Polyphaga includes the superfamilies Hydrophiloidea, Staphylinoidea, Cantharoidea, Cleroidea, Elateroidea, Dascilloidea, Dryopoidea, Byrrhoidea, Cucujoidea, Meloidea, Mordelloidea, Tenebrionoidea, Bostrichoidea, Scarabaeoidea, Cerambycoidea, Chrysomeloidea, and Curculionoidea. Superfamily Caraboidea includes the families Cicindelidae, Carabidae, and Dytiscidae. Superfamily Gyrinoidea includes the family Gyrinidae. Superfamily Hydrophiloidea includes the family Hydrophilidae. Superfamily Staphylinoidea includes the families Silphidae and Staphylinidae. Superfamily Cantharoidea includes the families Cantharidae and Lampyridae. Superfamily Cleroidea includes the families Cleridae and Dermestidae. Superfamily Elateroidea includes the families Elateridae and Buprestidae. Superfamily Cucujoidea includes the family Coccinellidae. Superfamily Meloidea includes the family Meloidae. Superfamily Tenebrionoidea includes the family Tenebrionidae. Superfamily Scarabaeoidea includes the families Passalidae and Scarabaeidae. Superfamily Cerambycoidea includes the family Cerambycidae. Superfamily Chrysomeloidea includes the family Chrysomelidae. Superfamily Curculionoidea includes the families Curculionidae and Scolytidae.

The order Diptera includes the Suborders Nematocera, Brachycera, and Cyclorrhapha. Suborder Nematocera includes the families Tipulidae, Psychodidae, Culicidae, Ceratopogonidae, Chironomidae, Simuliidae, Bibionidae, and Cecidomyiidae. Suborder Brachycera includes the families Stratiomyidae, Tabanidae, Therevidae, Asilidae, Mydidae, Bombyliidae, and Dolichopodidae. Suborder Cyclorrhapha includes the Divisions Aschiza and Aschiza. Division Aschiza includes the families Phoridae, Syrphidae, and Conopidae. Division Aschiza includes the Sections Acalyptratae and Calyptratae. Section Acalyptratae includes the families Otitidae, Tephritidae, Agromyzidae, and Drosophilidae. Section Calyptratae includes the families Hippoboscidae, Oestridae, Tachinidae, Anthomyiidae, Muscidae, Calliphoridae, and Sarcophagidae.

The order Lepidoptera includes the families Papilionidae, Pieridae, Lycaenidae, Nymphalidae, Danaidae, Satyridae, Hesperiidae, Sphingidae, Saturniidae, Geometridae, Arctiidae, Noctuidae, Lymantriidae, Sesiidae, and Tineidae.

Insect pests of the invention for the major crops include: Maize: Ostrinia nubilalis, European corn borer; Agrotis ipsilon, black cutworm; Helicoverpa zea, corn earworm; Spodoptera frugiperda, fall armyworm; Diatraea grandiosella, southwestern corn borer; Elasmopalpus lignosellus, lesser cornstalk borer; Diatraea saccharalis, surgarcane borer; Diabrotica virgifera, western corn rootworm; Diabrotica longicornis barberi, northern corn rootworm; Diabrotica undecimpunctata howardi, southern corn rootworm; Melanotus spp., wireworms; Cyclocephala borealis, northern masked chafer (white grub); Cyclocephala immaculata, southern masked chafer (white grub); Popillia japonica, Japanese beetle; Chaetocnema pulicaria, corn flea beetle; Sphenophorus maidis, maize billbug; Rhopalosiphum maidis, corn leaf aphid; Anuraphis maidiradicis, corn root aphid; Blissus leucopterus leucopterus, chinch bug; Melanoplus femurrubrum, redlegged grasshopper; Melanoplus sanguinipes, migratory grasshopper; Hylemya platura, seedcorn maggot; Agromyza parvicornis, corn blot leafminer; Anaphothrips obscrurus, grass thrips; Solenopsis milesta, thief ant; Tetranychus urticae, twospotted spider mite; Sorghum: Chilo partellus, sorghum borer; Spodoptera frugiperda, fall armyworm; Helicoverpa zea, corn earworm; Elasmopalpus lignosellus, lesser cornstalk borer; Feltia subterranea, granulate cutworm; Phyllophaga crinita, white grub; Eleodes, Conoderus, and Aeolus spp., wireworms; Oulema melanopus, cereal leaf beetle; Chaetocnema pulicaria, corn flea beetle; Sphenophorus maidis, maize billbug; Rhopalosiphum maidis; corn leaf aphid; Sipha flava, yellow sugarcane aphid; Blissus leucopterus leucopterus, chinch bug; Contarinia sorghicola, sorghum midge; Tetranychus cinnabarinus, carmine spider mite; Tetranychus urticae, twospotted spider mite; Wheat: Pseudaletia unipunctata, army worm; Spodoptera frugiperda, fall armyworm; Elasmopalpus lignosellus, lesser cornstalk borer; Agrotis orthogonia, western cutworm; Elasmopalpus lignosellus, lesser cornstalk borer; Oulema melanopus, cereal leaf beetle; Hypera punctata, clover leaf weevil; Diabrotica undecimpunctata howardi, southern corn rootworm; Russian wheat aphid; Schizaphis graminum, greenbug; Macrosiphum avenae, English grain aphid; Melanoplus femurrubrum, redlegged grasshopper; Melanoplus differentialis, differential grasshopper; Melanoplus sanguinipes, migratory grasshopper; Mayetiola destructor, Hessian fly; Sitodiplosis mosellana, wheat midge; Meromyza americana, wheat stem maggot; Hylemya coarctata, wheat bulb fly; Frankliniella fusca, tobacco thrips; Cephus cinctus, wheat stem sawfly; Aceria tulipae, wheat curl mite; Sunflower: Suleima helianthana, sunflower bud moth; Homoeosoma electellum, sunflower moth; zygogramma exclamationis, sunflower beetle; Bothyrus gibbosus, carrot beetle; Neolasioptera murtfeldtiana, sunflower seed midge; Cotton: Heliothis virescens, cotton budworm; Helicoverpa zea, cotton bollworm; Spodoptera exigua, beet armyworm; Pectinophora gossypiella, pink bollworm; Anthonomus grandis, boll weevil; Aphis gossypii, cotton aphid; Pseudatomoscelis seriatus, cotton fleahopper; Trialeurodes abutilonea, bandedwinged whitefly; Lygus lineolaris, tarnished plant bug; Melanoplus femurrubrum, redlegged grasshopper; Melanoplus differentialis, differential grasshopper; Thrips tabaci, onion thrips; Franklinkiella fusca, tobacco thrips; Tetranychus cinnabarinus, carmine spider mite; Tetranychus urticae, twospotted spider mite; Rice: Diatraea saccharalis, sugarcane borer; Spodoptera frugiperda, fall armyworm; Helicoverpa zea, corn earworm; Colaspis brunnea, grape colaspis; Lissorhoptrus oryzophilus, rice water weevil; Sitophilus oryzae, rice weevil; Nephotettix nigropictus, rice leafhopper; Blissus leucopterus leucopterus, chinch bug; Acrosternum hilare, green stink bug; Soybean: Pseudoplusia includens, soybean looper; Anticarsia gemmatalis, velvetbean caterpillar; Plathypena scabra, green cloverworm; Ostrinia nubilalis, European corn borer; Agrotis ipsilon, black cutworm; Spodoptera exigua, beet armyworm; Heliothis virescens, cotton budworm; Helicoverpa zea, cotton bollworm; Epilachna varivestis, Mexican bean beetle; Myzus persicae, green peach aphid; Empoasca fabae, potato leafhopper; Acrosternum hilare, green stink bug; Melanoplus femurrubrum, redlegged grasshopper; Melanoplus differentialis, differential grasshopper; Hylemya platura, seedcorn maggot; Sericothrips variabilis, soybean thrips; Thrips tabaci, onion thrips; Tetranychus turkestani, strawberry spider mite; Tetranychus urticae, twospotted spider mite; Barley: Ostrinia nubilalis, European corn borer; Agrotis ipsilon, black cutworm; Schizaphis graminum, greenbug; Blissus leucopterus leucopterus, chinch bug; Acrosternum hilare, green stink bug; Euschistus servus, brown stink bug; Delia platura, seedcorn maggot; Mayetiola destructor, Hessian fly; Petrobia latens, brown wheat mite; Oil Seed Rape: Brevicoryne brassicae, cabbage aphid; Phyllotreta cruciferae, Flea beetle; Mamestra configurata, Bertha armyworm; Plutella xylostella, Diamond-back moth; Delia ssp., Root maggots.

Nematodes include parasitic nematodes such as root-knot, cyst, and lesion nematodes, including Heterodera spp., Meloidogyne spp., and Globodera spp.; particularly members of the cyst nematodes, including, but not limited to, Heterodera glycines (soybean cyst nematode); Heterodera schachtii (beet cyst nematode); Heterodera avenae (cereal cyst nematode); and Globodera rostochiensis and Globodera pailida (potato cyst nematodes). Lesion nematodes include Pratylenchus spp.

### Methods for Increasing Plant Yield

Methods for increasing plant yield are provided. The methods comprise introducing into a plant or plant cell a polynucleotide comprising a pesticidal sequence disclosed herein. As defined herein, the “yield” of the plant refers to the quality and/or quantity of biomass produced by the plant. By “biomass” is intended any measured plant product. An increase in biomass production is any improvement in the yield of the measured plant product. Increasing plant yield has several commercial applications. For example, increasing plant leaf biomass may increase the yield of leafy vegetables for human or animal consumption. Additionally, increasing leaf biomass can be used to increase production of plant-derived pharmaceutical or industrial products. An increase in yield can comprise any statistically significant increase including, but not limited to, at least a 1% increase, at least a 3% increase, at least a 5% increase, at least a 10% increase, at least a 20% increase, at least a 30%, at least a 50%, at least a 70%, at least a 100% or a greater increase in yield compared to a plant not expressing the pesticidal sequence.

The following examples are offered by way of illustration and not by way of limitation.

### EXPERIMENTAL

### Example 1

### Extraction of Plasmid DNA

A pure culture of strain ATX13046 was grown in large quantities of rich media. The culture was spun to harvest the cell pellet. The cell pellet was then prepared by treatment with SDS by methods known in the art, resulting in breakage of the cell wall and release of DNA. Proteins and large genomic DNA were then precipitated by a high salt concentration. The plasmid DNA was then precipitated by standard ethanol precipitation. The plasmid DNA was separated from any remaining chromosomal DNA by high-speed centrifugation through a cesium chloride gradient. The DNA was visualized in the gradient by UV light and the band of lower density (i.e. the lower band) was extracted using a syringe. This band contained the plasmid DNA from Strain ATX13046. The quality of the DNA was checked by visualization on an agarose gel.

### Example 2

### Cloning of Genes

The purified plasmid DNA was sheared into 5-10 kb sized fragments and the 5′ and 3′ single stranded overhangs repaired using T4 DNA polymerase and Klenow fragment in the presence of all four dNTPs. Phosphates were then attached to the 5′ ends by treatment with T4 polynucleotide kinase. The repaired DNA fragments were then ligated overnight into a standard high copy vector (i.e. pBluescript SK+), suitably prepared to accept the inserts as known in the art (for example by digestion with a restriction enzyme producing blunt ends).

The quality of the library was analyzed by digesting a subset of clones with a restriction enzyme known to have a cleavage site flanking the cloning site. A high percentage of clones were determined to contain inserts, with an average insert size of 5-6 kb.

### Example 3

### High Throughput Sequencing of Library Plates

Once the clone library quality was checked and confirmed, colonies were grown in a rich broth in 2 ml 96-well blocks overnight at 37° C. at a shaking speed of 350 rpm. The blocks were spun to harvest the cells to the bottom of the block. The blocks were then prepared by standard alkaline lysis prep in a high throughput format.

The end sequences of clones from this library were then determined for a large number of clones from each block in the following way: The DNA sequence of each clone chosen for analysis was determined using the fluorescent dye terminator sequencing technique (Applied Biosystems) and standard primers flanking each side of the cloning site. Once the reactions had been carried out in the thermocycler, the DNA was precipitated using standard ethanol precipitation. The DNA was resuspended in water and loaded onto a capillary sequencing machine. Each library plate of DNA was sequenced from either end of the cloning site, yielding two reads per plate over each insert.

### Example 4

### Assembly and Screening of Sequencing Data

DNA sequences obtained were compiled into an assembly project and aligned together to form contigs. This can be done efficiently using a computer program, such as Vector NTI, or alternatively by using the Phred/Phrap suite of DNA alignment and analysis programs. These contigs, along with any individual read that may not have been added to a contig, were compared to a compiled database of all classes of known pesticidal genes. Contigs or individual reads identified as having identity to a known endotoxin or pesticidal gene were analyzed further. Among the sequences obtained, DNA clones were identified as having homology to known endotoxin genes. Therefore, these clones were selected for further sequencing.

### Example 5

### Cloning of axmi-076

A fragment of DNA with homology to endotoxin genes was identified from Strain ATX14775. The full open reading frame was identified by Tail (Thermal Asymmetric Interlaced) PCR based methods as known in the art. Finally, using the DNA sequence of the full length open reading frame from the Tail (Thermal Asymmetric Interlaced) PCR product, the open reading frame was amplified directly by PCR from strain ATX14775 and cloned into a vector.

### Example 6

### Sequencing of Clones

Primers were designed to anneal to the clones of interest in a manner such that DNA sequences generated from such primers will overlap existing DNA sequence of the clone(s). This process, known as “oligo walking,” is well known in the art. This process was utilized to determine the entire DNA sequence of the region exhibiting homology to a known endotoxin gene. In the case of the genes of the invention, this process was used to determine the DNA sequence of the entire open reading frame, resulting in a single nucleotide sequence for each. The completed DNA sequence was then placed back into the original large assembly for further validation. This allowed incorporation of more DNA sequence reads into the contig, resulting in multiple reads of coverage over the entire region.

Analysis of the DNA sequence of each clone by methods known in the art identified an open reading frame on each insert with homology to known delta endotoxin genes. The open reading frames were designated as axmi-066 and axmi-076, respectively. The DNA sequence of axmi-066 is provided in SEQ ID NO:1, and the amino acid sequence of the predicted protein is provided in SEQ ID NO:2. The DNA sequence of axmi-076 is provided in SEQ ID NO:4 and its predicted protein sequence is provided in SEQ ID NO:5.

The predicted open reading frame of axmi-066 is 637 amino acids long. However, alignment with its closest endotoxin homologs suggests that the start of translation of axmi-066 in Bacillus is likely to be at the internal ATG start codon thirty nine nucleotides downstream of the first ATG start codon (corresponding to nucleotide position 39 of SEQ ID NO:1). This coding sequence is set forth in SEQ ID NO:9. Translational initiation at this internal start codon will result in a 624 amino acid protein with a molecular weight of 71 kD (SEQ ID NO:10). A possible but strong Shine-Dalgarno sequence is present 6 nucleotides upstream of this internal start codon, which supports the results of protein alignments.

### Example 7

### Synthetic Nucleotide Sequences Encoding AXMI-066 and AXMI-076

The optaxmi-066 gene (SEQ ID NO:3) represents a synthetic nucleotide sequence that upon translation, will encode the AXMI-066 protein.

The optaxmi-076 gene (SEQ ID NO:6) and the optaxmi-076v04 (SEQ ID NO:11) gene represent synthetic nucleotide sequences that upon translation, will encode the AXMI-076 protein.

### Example 8

### Homology of AXMI-066 and AXMI-076 to Known Endotoxin Genes

A search of DNA and protein databases with the DNA sequences and amino acid sequences of AXMI-066 and AXMI-076 revealed that they are homologous to known delta-endotoxin proteins.

FIG. 2 shows an alignment of AXMI-066 with several endotoxins. Blast searches identified members of the cry2 endotoxin family as having the strongest homology to AXMI-066. Alignment of the AXMI-066 protein (SEQ ID NO:2) to a large set of endotoxin proteins confirmed that AXMI-066 has 74.8% identity to the Cry2Aa1 toxin.

Alignment of the AXMI-076 protein (SEQ ID NO:5) to a large set of endotoxin proteins confirmed that AXMI-076 has 93.1% identity to the Cry2Ae1 toxin and 91.0% identity to the Cry2Aa1 toxin.

### Example 9

### Expression of Synthetic Sequences in Bacillus

The axmi-066, optaxmi-066, axmi-076, optaxmi-076v, or optaxmi-076v04 sequences (SEQ ID NO:1, 3, 4, 6, and 11, respectively) are amplified by PCR and cloned into the Bacillus expression vector such as pAX916 by methods well known in the art. The resulting clone is assayed for expression of the AXMI protein after transformation into cells of a cry(−) Bacillus thuringiensis strain. A Bacillus strain containing the axmi clone and expressing the AXMI insecticidal protein is grown in, for example, CYS media (10 g/l Bacto-casitone; 3 g/l yeast extract; 6 g/l KH₂PO₄; 14 g/l K₂HPO₄; 0.5 mM MgSO₄; 0.05 mM MnCl₂; 0.05 mM FeSO₄), until sporulation is evident by microscopic examination. Samples are prepared, and analyzed by polyacrylamide gel electrophoresis (PAGE).

In the case of AXMI-066, the axmi066 open reading frame starting from the internal ATG (corresponding to nucleotide positions 39-41 of SEQ ID NO:1) was amplified by PCR. The product was cloned into a Bacillus vector based on pAX916 as well as E. coli expression vector based on pRSF1b (Invitrogen). The resulting clones were confirmed by restriction analysis and finally by complete sequencing of the cloned gene. The resulting constructs are called pAX2755 and pAX2757 respectively.

### Example 10

### Insecticidal Activity of AXMI-066 and AXMI-076

AXMI-066 and AXMI-076 were tested for activity against important lepidopteran pests by bioassay. Cultures of a Bacillus strain containing pAX2755 were grown to sporulation, pelleted, and tested on insect pests with appropriate controls. In these tests AXMI-066 and AXMI-076 demonstrated activity on several Lepidopteran pests, as summarized Table 1.

TABLE 1
Insect activity of AXMI-066 and AXMI-076

| Insect | AXMI-066 | AXMI-076 |
|---|---|---|
| European corn borer | +/− | ++++ |
| (Ostrinia nubilalis) | | |
| Corn earworm | ++ | +++ |
| (Helicoverpa zea) | | |
| Tobacco budworm | ++ | ++++ |
| (Heliothis virescens) | | |
| Fall armyworm | ++ | ++ |
| (Spodoptera frugiperda) | | |
| Velvetbean caterpillar | +++ | ++++ |
| (Anticarsia gemmatalis) | | |
| Black cutworm | − | − |
| (Agrotis ipsilon) | | |

### Example 11

### AXMI-066 Variants

Alignment of the AXMI-066 (SEQ ID NO:10) and Cry2Aa protein sequences indicates that the apical loops 1 and 2 of axmi-066 are both 2 amino acids shorter than loops 1 and 2 of Cry2Aa. AXMI-066 contains an additional loop 3 that is missing in Cry2Aa. Variant libraries of AXMI-066 have been generated containing insertions of 2 amino acids in loops 1 and 2, respectively. A deletion of loop 3 in AXMI-066 has also been generated. AXMI-066 variants have been expressed and assayed for insecticidal activity on several Lepidopteran insects. Eleven AXMI-066 variants carrying insertions into loop 2 containing glycines have been identified that are active on several Lepidopteran insects.

Variant libraries of axmi-66 were generated using the Quickchange Lightening kit (Stratagene). Construct pAX5435 (His6-axmi-66 in pRSF1b) was mutagenized. The 2 libraries consist of 2 codon insertions between Val320 and Pro321 (loop 1) and Gly378 and Asp379 (loop 2), respectively. Each library contains permutations of all 64 codons for the two inserted positions. A deletion of loop 3 was also carried out. The mutagenic sense oligos are as follows:

Loop 1:

(SEQ ID NO:21)

1. CTTCCTTCGGCGTGNWNNWNCCCATCCTCGGCGGC

(SEQ ID NO:22)

2. CTTCCTTCGGCGTGNSNNSNCCCATCCTCGGCGGC

(SEQ ID NO:23)

3. CTTCCTTCGGCGTGNWNNSNCCCATCCTCGGCGGC

(SEQ ID NO:24)

4. CTTCCTTCGGCGTGNSNNWNCCCATCCTCGGCGGC
Loop 2:

(SEQ ID NO:25)

1. CGGCGTCTACAGAGGANWNNWNGATCTTCAGCACAACTGG

(SEQ ID NO:26)

2. CGGCGTCTACAGAGGANSNNSNGATCTTCAGCACAACTGG

(SEQ ID NO:27)

3. CGGCGTCTACAGAGGANSNNWNGATCTTCAGCACAACTGG

(SEQ ID NO:28)

4. CGGCGTCTACAGAGGANWNNSNGATCTTCAGCACAACTGG
Loop 3:

(SEQ ID NO:29)

CGCCTTCCTCCTCTCAGTGAAGAGCAACTACTTCC

Mutagenesis reactions contain a sense oligo as described above and the corresponding antisense oligo. The libraries were cloned, and a number of clones were sequenced. Clones selected for functional characterization contained various combinations of positively charged, negatively charged, aromatic, polar and apolar amino acids. The selected insertion variants were expressed in E. coli and soluble extracts were prepared by bead beating in 50 mM Na-Carbonate pH 10.5, 1 mM DTT. The extracts were assayed for activity against Corn Earworm “Hz” (Helicoverpa zea), European Corn Borer “ECB” (Ostrinia nubilalis), Tobacco budworm “Hv” (Heliothis virescens), Fall Armyworm “FAW” (Spodoptera frugiperda), Black Cutworm “BCW” (Agrotis ipsilon), and Velvetbean caterpillar “VBC” (Anticarsia gemmatalis). Loop 2 insertion variants containing glycines were active against lepidopteran insects. The loop 2 GT insertion showed the highest toxicity of the variants tested. No activity was detected in the loop 1 insertion variants and the loop 3 deletion variants.

Single point mutations of AXMI-066 (SEQ ID NO:10) were also created and tested against lepidopteran pests. Table 2 lists the position and mutations that resulted in polypeptides having pesticidal activity equal to or greater than the pesticidal activity SEQ ID NO:10.

TABLE 2

| Position Relative to | Residue in | |
|---|---|---|
| SEQ ID NO: 10 | SEQ ID NO: 10 | Active Mutants |
| 83 | P | T |
| 250 | L | I |
| 319 | G | K, F |
| 322 | I | S, V, Q, A |
| 323 | L | F |
| 376 | Y | N, I, R, S, V, A |
| 377 | R | E, Q, L |
| 378 | G | S, A, W |
| 379 | D | V, E |
| 380 | L | M, P, Y |
| 381 | Q | L |
| 401 | L | I |
| 406 | M | H, V, K, E, T, S, A, V, N |
| 407 | F | W, R |

### Example 12

### Assays for Pesticidal Activity

The axmi-066, optaxmi-066, axmi-076, and optaxmi-076 nucleotide sequences of the invention can be tested for their ability to produce pesticidal proteins. The ability of a pesticidal protein to act as a pesticide upon a pest is often assessed in a number of ways. One way well known in the art is to perform a feeding assay. In such a feeding assay, one exposes the pest to a sample containing either compounds to be tested or control samples. Often this is performed by placing the material to be tested, or a suitable dilution of such material, onto a material that the pest will ingest, such as an artificial diet. The material to be tested may be composed of a liquid, solid, or slurry. The material to be tested may be placed upon the surface and then allowed to dry. Alternatively, the material to be tested may be mixed with a molten artificial diet, then dispensed into the assay chamber. The assay chamber may be, for example, a cup, a dish, or a well of a microtiter plate.

Assays for sucking pests (for example aphids) may involve separating the test material from the insect by a partition, ideally a portion that can be pierced by the sucking mouth parts of the sucking insect, to allow ingestion of the test material. Often the test material is mixed with a feeding stimulant, such as sucrose, to promote ingestion of the test compound.

Other types of assays can include microinjection of the test material into the mouth, or gut of the pest, as well as development of transgenic plants, followed by test of the ability of the pest to feed upon the transgenic plant. Plant testing may involve isolation of the plant parts normally consumed, for example, small cages attached to a leaf, or isolation of entire plants in cages containing insects.

Other methods and approaches to assay pests are known in the art, and can be found, for example in Robertson and Preisler, eds. (1992) Pesticide bioassays with arthropods, CRC, Boca Raton, Fla. Alternatively, assays are commonly described in the journals Arthropod Management Tests and Journal of Economic Entomology or by discussion with members of the Entomological Society of America (ESA).

### Example 13

### Vectoring of Genes for Plant Expression

The coding regions of the invention are connected with appropriate promoter and terminator sequences for expression in plants. Such sequences are well known in the art and may include the rice actin promoter or maize ubiquitin promoter for expression in monocots, the Arabidopsis UBQ3 promoter or CaMV 35S promoter for expression in dicots, and the nos or PinII terminators. Techniques for producing and confirming promoter-gene-terminator constructs also are well known in the art.

In one aspect of the invention, synthetic DNA sequences are designed and generated. These synthetic sequences have altered nucleotide sequence relative to the parent sequence, but encode proteins that are essentially identical to the parent AXMI-066 or AXMI-076 protein (e.g., SEQ ID NO:3 or 6).

In another aspect of the invention, modified versions of the synthetic genes are designed such that the resulting peptide is targeted to a plant organelle, such as the endoplasmic reticulum or the apoplast. Peptide sequences known to result in targeting of fusion proteins to plant organelles are known in the art. For example, the N-terminal region of the acid phosphatase gene from the White Lupin Lupinus albus (GENBANK® ID GI: 14276838, Miller et al. (2001) Plant Physiology 127: 594-606) is known in the art to result in endoplasmic reticulum targeting of heterologous proteins. If the resulting fusion protein also contains an endoplasmic reticulum retention sequence comprising the peptide N-terminus-lysine-aspartic acid-glutamic acid-leucine (i.e., the “KDEL” motif, SEQ ID NO:7) at the C-terminus, the fusion protein will be targeted to the endoplasmic reticulum. If the fusion protein lacks an endoplasmic reticulum targeting sequence at the C-terminus, the protein will be targeted to the endoplasmic reticulum, but will ultimately be sequestered in the apoplast.

Thus, this gene encodes a fusion protein that contains the N-terminal thirty-one amino acids of the acid phosphatase gene from the White Lupin Lupinus albus (GENBANK® ID GI: 14276838, Miller et al., 2001, supra) fused to the N-terminus of the AXMI-066 or AXMI-076 sequence, as well as the KDEL sequence at the C-terminus. Thus, the resulting protein is predicted to be targeted the plant endoplasmic reticulum upon expression in a plant cell.

A construct comprising a nucleotide sequence encoding a chloroplast transit peptide derived from Chlamydomonas reinhardtii linked to the optaxmi-076v04 sequence is set forth in SEQ ID NO:12 (nucleotide sequence) and SEQ ID NO:13 (amino acid sequence).

The plant expression cassettes described above are combined with an appropriate plant selectable marker to aid in the selection of transformed cells and tissues, and ligated into plant transformation vectors. These may include binary vectors from Agrobacterium-mediated transformation or simple plasmid vectors for aerosol or biolistic transformation.

### Example 14

### Vectoring of axmi-066, optaxmi-066, axmi-076, and optaxmi-076 Genes for Plant Expression

The coding region DNA of the axmi-066, optaxmi-066, axmi-076, and optaxmi-076 genes of the invention are operably connected with appropriate promoter and terminator sequences for expression in plants. Such sequences are well known in the art and may include the rice actin promoter or maize ubiquitin promoter for expression in monocots, the Arabidopsis UBQ3 promoter or CaMV 35S promoter for expression in dicots, and the nos or PinII terminators. Techniques for producing and confirming promoter-gene-terminator constructs also are well known in the art.

The plant expression cassettes described above are combined with an appropriate plant selectable marker to aid in the selections of transformed cells and tissues, and ligated into plant transformation vectors. These may include binary vectors from Agrobacterium-mediated transformation or simple plasmid vectors for aerosol or biolistic transformation.

### Example 15

### Transformation of Maize Cells with the Pesticidal Protein Genes Described Herein

Maize ears are best collected 8-12 days after pollination. Embryos are isolated from the ears, and those embryos 0.8-1.5 mm in size are preferred for use in transformation. Embryos are plated scutellum side-up on a suitable incubation media, such as DN62A5S media (3.98 g/L N6 Salts; 1 mL/L (of 1000× Stock) N6 Vitamins; 800 mg/L L-Asparagine; 100 mg/L Myo-inositol; 1.4 g/L L-Proline; 100 mg/L Casamino acids; 50 g/L sucrose; 1 mL/L (of 1 mg/mL Stock) 2,4-D). However, media and salts other than DN62A5S are suitable and are known in the art. Embryos are incubated overnight at 25° C. in the dark. However, it is not necessary per se to incubate the embryos overnight.

The resulting explants are transferred to mesh squares (30-40 per plate), transferred onto osmotic media for about 30-45 minutes, then transferred to a beaming plate (see, for example, PCT Publication No. WO/0138514 and U.S. Pat. No. 5,240,842).

DNA constructs designed to the genes of the invention in plant cells are accelerated into plant tissue using an aerosol beam accelerator, using conditions essentially as described in PCT Publication No. WO/0138514. After beaming, embryos are incubated for about 30 min on osmotic media, and placed onto incubation media overnight at 25° C. in the dark. To avoid unduly damaging beamed explants, they are incubated for at least 24 hours prior to transfer to recovery media. Embryos are then spread onto recovery period media, for about 5 days, 25° C. in the dark, then transferred to a selection media. Explants are incubated in selection media for up to eight weeks, depending on the nature and characteristics of the particular selection utilized. After the selection period, the resulting callus is transferred to embryo maturation media, until the formation of mature somatic embryos is observed. The resulting mature somatic embryos are then placed under low light, and the process of regeneration is initiated by methods known in the art. The resulting shoots are allowed to root on rooting media, and the resulting plants are transferred to nursery pots and propagated as transgenic plants.

Materials
DN62A5S Media

| Components | Per Liter | | Source |
|---|---|---|---|
| Chu's N6 Basal Salt Mixture | 3.98 | g/L | Phytotechnology Labs |
| (Prod. No. C 416) | | | |
| Chu's N6 Vitamin Solution | 1 mL/L (of | | Phytotechnology Labs |
| (Prod. No. C 149) | 1000x Stock) | | |
| L-Asparagine | 800 | mg/L | Phytotechnology Labs |
| Myo-inositol | 100 | mg/L | Sigma |
| L-Proline | 1.4 | g/L | Phytotechnology Labs |
| Casamino acids | 100 | mg/L | Fisher Scientific |
| Sucrose | 50 | g/L | Phytotechnology Labs |

| | | |
|---|---|---|
| 2,4-D (Prod. No. D-7299) | 1 mL/L (of | Sigma |
| | 1 mg/mL Stock) | |

The pH of the solution is adjusted to pH 5.8 with 1N KOH/1N KCl, Gelrite (Sigma) is added at a concentration up to 3 g/L, and the media is autoclaved. After cooling to 50° C., 2 ml/L of a 5 mg/ml stock solution of silver nitrate (Phytotechnology Labs) is added.

### Example 16

### Transformation of the axmi-066, optaxmi-066, axmi-076, and optaxmi-076 Genes of the Invention in Plant Cells by Agrobacterium-Mediated Transformation

Ears are best collected 8-12 days after pollination. Embryos are isolated from the ears, and those embryos 0.8-1.5 mm in size are preferred for use in transformation. Embryos are plated scutellum side-up on a suitable incubation media, and incubated overnight at 25° C. in the dark. However, it is not necessary per se to incubate the embryos overnight. Embryos are contacted with an Agrobacterium strain containing the appropriate vectors for Ti plasmid mediated transfer for about 5-10 min, and then plated onto co-cultivation media for about 3 days (25° C. in the dark). After co-cultivation, explants are transferred to recovery period media for about five days (at 25° C. in the dark). Explants are incubated in selection media for up to eight weeks, depending on the nature and characteristics of the particular selection utilized. After the selection period, the resulting callus is transferred to embryo maturation media, until the formation of mature somatic embryos is observed. The resulting mature somatic embryos are then placed under low light, and the process of regeneration is initiated as known in the art.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. An isolated nucleic acid molecule comprising a nucleotide sequence encoding an amino acid sequence having pesticidal activity, wherein said nucleotide sequence is selected from the group consisting of:
a) the nucleotide sequence set forth in SEQ ID NO:4, 1, 3, 4, 6, 9, or 11;
b) a nucleotide sequence having at least 90% sequence identity to the nucleotide sequence of SEQ ID NO:4, 1, 3, 4, 6, 9, or 11;
c) a nucleotide sequence having at least 95% sequence identity to the nucleotide sequence of SEQ ID NO:4;
d) a nucleotide sequence that encodes a polypeptide comprising the amino acid sequence of SEQ ID NO:5, 2, or 10;
e) a nucleotide sequence that encodes a polypeptide comprising an amino acid sequence having at least 90% sequence identity to the amino acid sequence of SEQ ID NO:2 or 10;
f) a nucleotide sequence that encodes a polypeptide comprising an amino acid sequence having at least 95% sequence identity to the amino acid sequence of SEQ ID NO:5; and,
g) the nucleotide sequence of the DNA insert of the plasmid deposited as Accession No. B-50045.

2. The nucleic acid molecule of claim 1, wherein said nucleic acid molecule encodes a sequence having at least 90% sequence identity to the amino acid sequence set forth in SEQ ID NO:10, and wherein said amino acid sequence comprises an insertion of one or two amino acids between resides 379 and 380 of SEQ ID NO:10.

3. The nucleic acid molecule of claim 2, wherein said amino acid sequence comprises an insertion of two amino acids between resides 379 and 380 of SEQ ID NO:10, and wherein said insertion is selected from the group consisting of a glycine and a glycine, a glycine and a threonine, a glycine and a serine, a glycine and a leucine, an arginine and a glycine, a glycine and an asparagine, a glycine and a lysine, a histidine and a glycine, a phenylalanine and a glycine, a leucine and a glycine, and an asparagine and a glycine residue.

4. The isolated nucleic acid molecule of claim 1, wherein said nucleotide sequence is a synthetic sequence that has been designed for expression in a plant.

5. A vector comprising the nucleic acid molecule of claim 1.

6. The vector of claim 5, further comprising a nucleic acid molecule encoding a heterologous polypeptide.

7. A host cell that contains the vector of claim 5.

8. The host cell of claim 7 that is a bacterial host cell.

9. The host cell of claim 7 that is a plant cell.

10. A transgenic plant comprising the host cell of claim 9.

11. The transgenic plant of claim 10, wherein said plant is selected from the group consisting of maize, sorghum, wheat, cabbage, sunflower, tomato, crucifers, peppers, potato, cotton, rice, soybean, sugarbeet, sugarcane, tobacco, barley, and oilseed rape.

12. A transgenic seed comprising the nucleic acid molecule of claim 1.

13. An isolated polypeptide with pesticidal activity, selected from the group consisting of:
a) a polypeptide comprising the amino acid sequence of SEQ ID NO:5, 2, or 10;
b) a polypeptide comprising an amino acid sequence having at least 90% sequence identity to the amino acid sequence of SEQ ID NO:2 or 10;
c) a polypeptide comprising an amino acid sequence having at least 95% sequence identity to the amino acid sequence of SEQ ID NO:5;
d) a polypeptide that is encoded by SEQ ID NO:4, 1, 3, 4, 6, 9, or 11;
e) a polypeptide that is encoded by a nucleotide sequence that is at least 90% identical to the nucleotide sequence of SEQ ID NO:9, 1, 3, 6, or 11;
f) a polypeptide that is encoded by a nucleotide sequence that is at least 95% identical to the nucleotide sequence of SEQ ID NO:4; and,
f) a polypeptide encoded by the nucleotide sequence of the DNA insert of the plasmid deposited as Accession No. B-50045.

14. The polypeptide of claim 13, wherein said polypeptide comprises a sequence having at least 90% sequence identity to the amino acid sequence set forth in SEQ ID NO:10, and wherein said amino acid sequence comprises an insertion of one or two amino acids between resides 379 and 380 of SEQ ID NO:10.

15. The polypeptide of claim 14, wherein said amino acid sequence comprises an insertion of two amino acids between resides 379 and 380 of SEQ ID NO:10, and wherein said insertion is selected from the group consisting of a glycine and a glycine, a glycine and a threonine, a glycine and a serine, a glycine and a leucine, an arginine and a glycine, a glycine and an asparagine, a glycine and a lysine, a histidine and a glycine, a phenylalanine and a glycine, a leucine and a glycine, and an asparagine and a glycine residue.

16. The polypeptide of claim 13 further comprising heterologous amino acid sequences.

17. A composition comprising the polypeptide of claim 13.

18. The composition of claim 17, wherein said composition is selected from the group consisting of a powder, dust, pellet, granule, spray, emulsion, colloid, and solution.

19. The composition of claim 17, wherein said composition is prepared by desiccation, lyophilization, homogenization, extraction, filtration, centrifugation, sedimentation, or concentration of a culture of bacterial cells.

20. The composition of claim 17, comprising from about 1% to about 99% by weight of said polypeptide.

21. A method for controlling a lepidopteran, coleopteran, nematode, or dipteran pest population comprising contacting said population with a pesticidally-effective amount of a polypeptide of claim 13.

22. A method for killing a lepidopteran, coleopteran, nematode, or dipteran pest, comprising contacting said pest with, or feeding to said pest, a pesticidally-effective amount of a polypeptide of claim 13.

23. A method for producing a polypeptide with pesticidal activity, comprising culturing the host cell of claim 6 under conditions in which the nucleic acid molecule encoding the polypeptide is expressed.

24. A plant having stably incorporated into its genome a DNA construct comprising a nucleotide sequence that encodes a protein having pesticidal activity, wherein said nucleotide sequence is selected from the group consisting of:
a) the nucleotide sequence set forth in SEQ ID NO:4, 1, 3, 4, 6, 9, or 11;
b) a nucleotide sequence having at least 90% sequence identity to the nucleotide sequence of SEQ ID NO:4, 1, 3, 4, 6, 9, or 11;
c) a nucleotide sequence having at least 95% sequence identity to the nucleotide sequence of SEQ ID NO:4;
d) a nucleotide sequence that encodes a polypeptide comprising the amino acid sequence of SEQ ID NO:5, 2, or 10;
e) a nucleotide sequence that encodes a polypeptide comprising an amino acid sequence having at least 90% sequence identity to the amino acid sequence of SEQ ID NO:2 or 10;
f) a nucleotide sequence that encodes a polypeptide comprising an amino acid sequence having at least 95% sequence identity to the amino acid sequence of SEQ ID NO:5; and,
g) the nucleotide sequence of the DNA insert of the plasmid deposited as Accession No. B-50045;
wherein said nucleotide sequence is operably linked to a promoter that drives expression of a coding sequence in a plant cell.

25. The plant of claim 24, wherein said nucleic acid molecule encodes a sequence having at least 90% sequence identity to the amino acid sequence set forth in SEQ ID NO:10, and wherein said amino acid sequence comprises an insertion of one or two amino acids between resides 379 and 380 of SEQ ID NO:10.

26. The plant of claim 25, wherein said amino acid sequence comprises an insertion of two amino acids between resides 379 and 380 of SEQ ID NO:10, and wherein said insertion is selected from the group consisting of a glycine and a glycine, a glycine and a threonine, a glycine and a serine, a glycine and a leucine, an arginine and a glycine, a glycine and an asparagine, a glycine and a lysine, a histidine and a glycine, a phenylalanine and a glycine, a leucine and a glycine, and an asparagine and a glycine residue.

27. The plant of claim 24, wherein said plant is a plant cell.

28. A method for protecting a plant from a pest, comprising introducing into said plant or cell thereof at least one expression vector comprising a nucleotide sequence that encodes a pesticidal polypeptide, wherein said nucleotide sequence is selected from the group consisting of:
a) the nucleotide sequence set forth in SEQ ID NO:4, 1, 3, 4, 6, 9, or 11;
b) a nucleotide sequence having at least 90% sequence identity to the nucleotide sequence of SEQ ID NO:4, 1, 3, 4, 6, 9, or 11;
c) a nucleotide sequence having at least 95% sequence identity to the nucleotide sequence of SEQ ID NO:4;
d) a nucleotide sequence that encodes a polypeptide comprising the amino acid sequence of SEQ ID NO:5, 2, or 10;
e) a nucleotide sequence that encodes a polypeptide comprising an amino acid sequence having at least 90% sequence identity to the amino acid sequence of SEQ ID NO:2 or 10;
f) a nucleotide sequence that encodes a polypeptide comprising an amino acid sequence having at least 95% sequence identity to the amino acid sequence of SEQ ID NO:5; and,
g) the nucleotide sequence of the DNA insert of the plasmid deposited as Accession No. B-50045.

29. The method of claim 28, wherein said plant produces a pesticidal polypeptide having pesticidal activity against a lepidopteran, coleopteran, nematode, or dipteran pest.
